(19) United States
(12) Patent Application Publication
(10) Pub. No.: US 2002/0052323 A1
(43) Pub. Date: May 2, 2002

(54) Quinoline-(C=O)-(multiple amino acids)-leaving group compounds for pharmaceutical compositions and reagents

(76) Inventor: Jinhai Wang, Foster City, CA (US)

Correspondence Address: PETERS, VERNY, JONES & BIKSA LLP, 385 SHERMAN AVENUE, SUITE 6, PALO ALTO, CA, 94306, US

(21) Appl. No.: 09/870,027

(22) Filed: May 29, 2001

Related U.S. Application Data
(60) Provisional application No. 60/229,257, filed on Aug. 30, 2000.

(51) Int. Cl. A61K 38/05; C07D 215/38

(52) U.S. Cl. 514/19; 546/175

(57) ABSTRACT

This invention concerns compounds and a pharmaceutical composition of the structure:

wherein:

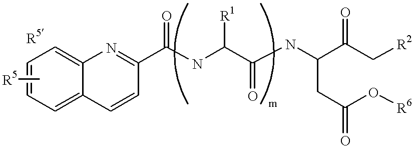

R¹ is selected from the group consisting of alkyl, substituted alkyl, aryl, and substituted aryl which group will produce a natural amino acid structure or an unnatural amino acid structure, and the carbon adjacent to R¹ is in the D or L configuration;

R² is selected from the group consisting of

—F

and

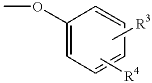

wherein R³ and R⁴ are each selected from the group consisting of hydrogen, alkyl, fluoro, chloro, carboxyl, alkoxy, alkyl carbonyl, aryl carbonyl, and amino; R⁵ and R^{5′} are each independently selected from the group consisting of hydrogen, alkyl, alkoxy, fluoro, chloro, carboxy, alkoxy, alkyl carbonyl, aryl carbonyl, and amino, R⁶ is selected from the group consisting of alkyl having 1 to 10 carbon atoms, aryl or substituted aryl, and m is 1, 2 or 3. These compounds are reagents and pharmaceutical compositions have pro-drug and apoptosis properties and are useful in a variety of therapies, for diseases such as arthritis, ALS, MS, and the like.

### RELATED APPLICATIONS

This application is a continuation-in-part application of U.S. Ser. No. 60/229,257, filed Aug. 30, 2000 which is incorporated herein by reference in its entirety.

### BACKGROUND OF THE INVENTION

1. Field of the Invention

This invention concerns substituted quinoline-(multiple-amino acids)-leaving group structures (e.g., substituted phenol or fluoromethyl ketone) (and quinoline-type structures) as novel compositions of matter. Two, three or four amino acid linking groups are described. These structures have a variety of therapeutic and pharmaceutical uses, including use as prodrugs and as protease inhibitors, particularly for caspase enzyme.

2. Description of Related Art

Some research in the use of protease inhibitors has been reported in the open literature and in the patent literature.

L. C. Fritz, et al. in U.S. Pat. No. 6,200,969 which recently issued on Mar. 13, 2001, describe methods and structures for expanding and increasing survival of hematopoietic cell population for prolonging viability of an organ for transplantation, and enhancing bioproduction using interlukin-1β-converting enzyme (ICE)/CED-3 family inhibitors. This patent does not teach or suggest the present invention.

D. H. Rasnick in U.S. Pat. No. 4,518,528 discloses novel α-amino fluoroketones, a method for their synthesis and a method for irreversibly inhibiting proteases. Quinoline structures are not taught or suggested.

M. P. Zimmerman, et al. in U.S. Pat. No. 5,714,484 disclose cysteine protease inhibitors which target a desired cysteine protease and positions the inhibitor near the thiolate anion portion of the active site of the protease. A second portion covalently bonds to the cysteine protease and irreversibly deactivates that protease by providing a carbonyl or carbonyl-equivalent. This structure is attacked by the thionate anion of the active site of the cysteine protease to sequentially cleave a β-carbonyl enol ether leaving group. Some of the fluoromethyl ketones were shown to be toxic upon chronic treatment and were not useful as potential drugs. The phenoxy ether compounds were reported to be not effective enough when compared to results reported as anti-caspase inhibitors.

The references of interest are listed here and are found referenced by in the text below by numbers in parentheses.

1. C. S. He, et al. 1989. Tissue co-operation in a proteolytic cascade activating human interstitial collagenase. Proc Natl Acad Sci USA, 86, 2632-6.

2. L. A. Liotta, et al. 1991. Cancer metastasis and angiogenesis: an imbalance of positive and negative regulation. Cell, 64, 327-36.

3. H. Kobayashi, et al. 1993. Effects of membrane-associated cathepsin B on the activation of receptor-bound pro-urokinase and subsequent invasion of reconstituted basement membranes. Biochem Biophys Acta, 1178, 55-62.

4. C. Ruppert, et al. 1994. Proteases associated with gynecological tumors. Int J Oncol 4, 717-21.

5. M. Sivaparvathi, et al. 1995. Overexpressison and localization of cathepsin B during the progression of human gliomas. Clin Exp Metastasis, 13,49-56.

6. L. A. Liotta, et al. 1991. Tumor invasion and metastasis; an imbalance of positive and negative regulation. Cancer Res. 51, 5054s-59s.

7. R. Reich, et al. 1988. Effects of inhibitors of plasminogen activator, serine proteinases, and collagenase IV on the invasion of basement membranes by metastatic cells. Cancer Res. 48, 3307-12.

8. P. Mignatti, et al. 1993. Biology and biochemistry of proteinases in tumor invasion. Physiol Rev. 73, 161-95.

9. W. L. Monsky, et al. 1994. A potential marker protease of invasiveness, seprase, is localized on invadopodia of human malignant melanoma cells. Cancer Res. 54, 5702-10.

10. L. A. Liotta, et al. 1980. Metastatic potential correlates with enzymatic degradation of basement membrane collagen. Nature, 284, 67-8.

11. M. R. Emmert-Buck, et al. 1994. Increased gelatinase A (MMP-2) and cathepsin B activity in invasive tumor regions of human colon cancer samples. Am J Pathol. 145, 1285-90.

12. L. Ossowki, et al. 1983. Antibodies to plasminogen activator inhibit human tumor metastasis. Cell, 35, 611-19.

13. P. Mignatti, et al. 1986. Tumor invasion through the human amniotic membrane; requirement for a proteinase cascade. Cell, 47, 487-98.

14. C. F. Sier, et al. 1994. Inactive urokinase and increased levels of its inhibitor type 1 in colorectal cancer liver metastasis. Gastroenterology, 107, 1449-56.

15. B. F. Sloane, et al. 1981. Lysosomal cathepsin B: correlation with metastatic potential. Science, 212, 1151-3.

16. B. F. Sloane, et a. 1984. Cysteine proteinases and metastasis. Cancer Metastasis Rev. 3, 249-63.

17. E. Elliott, et al. 1996. The cysteine protease cathepsin B in cancer. Perspect Drug Discov Design. 6, 12-32.

18. T. T. Lah, et al. 1995. Cathepsins D, B and L in breast carcinoma and in transformed human breast epithelial cells (HBEC). Biol Chem Hoppe-Seyler, 376, 357-63.

19. P. B. Scaddan, et al. 1993. Characterization of cysteine proteases and their endogenous inhibitors in MCF-7 and adriamycin-resistant MCF-7 human breast cancer cells. Inv Metastasis, 13, 301-13.

20. R. A. Maciewicz, et al. 1989. Immunodetection of cathepsins B and L present in and secreted from human premalignant and malignant colorectal tumor cell lines. Int J Cancer, 43, 478-86.

21. F. Oian, et al. 1989. Expression of five cathepsins in murine melanomas of varying metastatic potential and normal tissues. Cancer Res, 49,1870-5.

22. M. J. Murnane, et al. 1991. Stage-specific increases in cathepsin messenger RNA content in human colorectal carcinoma. Cancer Res, 51, 1137-42.

23. T. Inoue, et al. 1994. Cathepsin B expression and laminin degradation as factors influencing prognosis of surgically treated patients with lung adenocarcinoma. Cancer Res, 54, 6133-6.

24. S. A. Rempel, et al. 1994. Cathepsin B expression and localization in glioma progression and invasion. Cancer Res, 54, 6027-31.

25. E. Campo, et al. 1994. Cathepsin B expression in colorectal carcinomas correlates with tumor progression shortened patient survival. Am J Pathol, 145, 301-9.

26. K. Sheahan, et al. 1989. Cysteine protease activities and tumor development in human colorectal carcinoma. Cancer Res, 49, 3809-14.

27. V. Evers, et al. 1985. The digestion of phagocytosed collagen is inhibited by the proteinase inhibitors leupeptin and E-64. Collagen Rel Res, 5, 315-36.

28. C. J. F. Van Noorden, et al. 1991. Selective inhibition of cysteine proteinases by Z-Phe-Ala-CH₂F suppresses digestion of collagen by fibroblasts and osteoclasts. Biochem Biophys Res Commun, 178, 178-84.

29. B. F. Sloane, et al. 1986. Cathepsin B: association with plasma membrane in metastatic tumors. Proc Natl Acad Sci, USA, 83, 2483-7.

30. J. Rozhin, et al. 1994. Pericellular pH affects distribution and secretion of cathepsin B in malignant cells. Cancer Res, 54, 6517-25.

31. D. Keppler, et al. 1994. Secretion of cathepsin B and tumor invasion. Biochem Soc Trans, 22, 43-9.

32. V. Y. Reddy, et al. 1995. Pericellular mobilization of the tissue-destructive cysteine proteinases, cathpsins B,L, and S, by human monocyte-derived macrophages. Proc Natl Acad Sci USA, 92, 3849-53.

33. C. J. F. Van Noorden, et al. 1989. Localization and cytophotometric analysis of cathepsin B activity in unfixed and decalcified cryostat sections of whole rat knee joints. J Histochem Cytochem, 37, 617-24.

34. C. J. F. Van Noorden, et al. 1988. Cysteine proteinase activity in arthritic rat knee joints and the effects of a selective systemic inhibitor, Z-Phe-AlaCH₂F. J Rheumatol, 15, 1525-35.

35. M. Erdel, et al. 1990. Localization of cathepsin B in two human lung cancer cell lines. J Histochem Cytochem, 38, 1313-21.

36. E. Spiess, et al. 1994. Cathepsin B activity in human lung tumor cell lines; ultrastructural localization, pH sensitivity, and inhibitor status at the cellular level. J Histochem Cytochem, 42, 917-29.

37. S. J. Chan, et al. 1986. Nucleotide and predicted amino acid sequences of cloned human and mouse preprocathepsin B cDNAs. Proc Natl Acad Sci USA, 83, 7721-5.

38. D. Keppler, et al. 1994. Latency of cathepsin B secreted by human colon carcinoma cells is not linked to secretion of cystatin C and is relieved by neutrophil elastase. Biochem Biophys Acta, 1226, 117-25.

39. C. B. Basbaum, et al. 1996. Focalized proteolysis—spatial and temporal regulation of extracellular matrix degradation at the cell surface. Curr Opin Cell Biol, 8, 731-8.

40. R. E. Esser, et al. 1994. Cysteine proteinase inhibitors decrease articular cartilage and bone destruction in chronic inflammatory arthritis. Arth Rheum, 37, 236-47.

41. G. Harth, et al. 1993. Peptide-fluoromethyl ketones arrest intracellular replication and intercellular transmission of Trypanosomacruzi. Mol Biochem Parasitol, 58, 17-24.

42. C. C. Calkins, et al. 1995. Mammalian cysteine protease inhibitors; biochemical properties and possible roles in tumor progression. Biol Chem Hoppe Seyler, 376, 71-80.May 29, 2001

43. J. S. Mort, et al. 1986. Interrelationship of active and latent secreted human cathepsin B precursors. Biochem J, 233, 57-63.

44. R. L. Marquet, et al 1984. Interferon treatment of a transplantable rat colon adenocarcinoma; importance of tumor site. Int J Cancer, 33, 689-92.

45. K. P. Dingemans, et al. 1994. Developmental stages in experimental liver metastases; relation to invasiveness. Int J Cancer, 57, 433-9.

46. P. Griffini, et al. 1997. Three-dimensional reconstruction of colon carcinoma metastases in liver. J Microsc, 187, 12-21.

47. L. H. P. Caro, et al. 1988. 3-Methyladenine, an inhibitor of autophagy, has multiple effects on metabolism. Eur J Biochem, 175, 325-9.

48. C. J. F. Van Noorden, et al. 1997. Ala-Pro-cresyl violet, a synthetic fluorogenic substrate for the analysis of kinetic parameters of dipeptidyl peptidase IV (CD 26) in individual living rat hepatocytes. Anal Biochem.

49. A. Jonker, et al, 1995. Image analysis and image processing as tools to measure initial rates of enzyme reactions in sections: distribution patterns of glutamate dehydrogerase activity in rat liver lobules. J Histochem Cytochem, 43, 1027-34.

50. R. E. Wilson, et al. 1989. Enhanced synthesis of specific proteins, RNA, and DNA caused by hypoxia and reoxygenation. Int J Radiat Oncol, 16, 957-61.

51. S. M. Smorenburg, et al. 1996. Alpha-2-macroglobulin is mainly produced by cancer cells and not by hepatocytes in rats with colon carcinoma metastases in liver. Hepatology, 23, 560-70.

52. P. Griffini, et al. 1996. Kupffer cells and pit cells are not effective in the defense against experimentally induced colon carcinoma metastasis in rat liver. Clin Exp Metastasis, 14, 367-80.

53. N. K. Ahmed, et al. 1993. Peptidyl fluoromethyl ketones as inhibitors of cathepsin B. Biochem Pharmacol, 44, 1201-7.

54. C. J. F. Van Noorden, et al. 1992. Enzyme Histochemistry. A Laboratory Manual of Current Methods, Oxford. BIOS.

55. J. Caldero, et al. 1989. Lectin-binding sites in neoplastic and nonneoplastic colonic mucosa of 1,2-dimethylhydrazine-treated rats. Lab Invest, 61, 670-6.

56. S. W. Cox, et al. 1987. Preliminary studies on cysteine and serine proteinase activities in inflamed human gingiva using different 7-amino-4-trifluoromethyl coumarin substrates and protease inhibitors. Arch Oral Biol, 32, 599-605.

57. M. Pagano, et al. 1986. Inhibition of the cathepsin B like proteinase by a low molecular weight cysteine-proteinase inhibitor from ascitic fluid and plasma alpha 2 macroglobulin. Biochem Cell Biol, 64, 1218-25.

58. S. Yagel, et al. 1989. Suppression by cathepsin L inhibitors of the invasion of amnion membranes by murine cancer cells. Cancer Res, 49, 3553-7.

59. S. M. Redwood, et al. 1992. Abrogation of the invasion of human bladder tumor cells by using protease inhibitor(s). Cancer, 69, 1212-19.

60. R. Navab, et al. 1997. Inhibition of carcinoma cell invasion and liver metastases formation by the cysteine proteinase inhibitor E-64. Clin Exp Metastasis, 15, 121-9.

61. I. J. Fidler, 1991. Cancer metastasis. Br Med Bull, 47, 157-77.

62. I. J. Fidler, et al. 1994. Modulation of tumor cell response to chemotherapy by the organ environment. Cancer Metastasis Rev, 13, 209-22.

63. M. Nakajima, et al. 1990. Influence of organ environment on extracellular matrix degradative activity and metastasis of human colon carcinoma cells. J Natl Cancer Inst, 82, 1890-8.

64. A. M. Wheatley, et al. 1993. Interpretation of the laser Doppler flow signal from the liver of the rat. Microvasc Res, 45, 290-301.

65. F. C. Richardson, et al. 1988. Hepatocyte initiation during continuous administration of diethylnitrosamine and 1,2-sym-dimethylhydrazine. Cancer Res, 48,988-92.

66. L. W. Elmore, 1991. Phenotypic characterization of metaplastic intestinal glands and ductular hepatocyates in cholangiofibrotic lesions rapidly induced in the caudate liver lobe of rats treated with furan. Cancer Res, 51, 5752-9.

67. H. B. Jones, et al. 1993. Phenobarbital-induced hepatocellular proliferation: anti-bromodeoxyuridine and anti-proliferating cell nuclear antigen immunocytochemistry. J Histochem Cytochem, 41, 21-7.

68. Y. Shirai, et al. 1996. Colorectal caracinoma metastases to the liver does primary tumor location affect its lobar distribution. Cancer, 77, 2213-16.

69. W. J. Dodds, et al. 1990. Caudate lobe of the liver: anatomy, embryology and pathology. Am J Roentgenol, 154, 87-93.

70. E. Barbera-Guillem, et al. 1989. Selective implantation and growth in rats and mice of experimental liver metastasis in acinar zone one. Cancer Res, 49, 4003-10.

71. E. Barbera-Guillem, et al. 1991. Differences in the lectin-binding patterns of the periportal and perivenous endothelial domains in the liver sinusoids. Hepatology, 14, 131-9.

72. C. D. Dijkstra, et al. 1985. The heterogeneity of mononuclear phagocytes in lymphoid organs: distinct macarophage subpopulations in the rat recognized by monoclonal antibodies ED1, ED2 and ED3. Immunology, 54, 589-99.

73. W. R. McMaster, et al. 1979. Identification of Ia glycoproteins in rat thymus and purification from rat spleen. Eur J Immunol, 9, 426-33.

74. L. Bouwens, et al. 1992. Pit cells in the liver. Liver, 12, 3-9.

75. A. M. Duijvestijn, et al. 1992. Antibodies defining rat endothelial cells: RECA-1. A pan-endothelial cell-specific monoclonal antibody. Lab Invest, 66, 459-66.

76. A. P. Robinson, et al. 1986. MRC 0X43: a monoclonal antibody which reacts with all vascular endothelium in the rat except that of brain capillaries. Immunology, 57, 231-7.

77. E. N. Lamme, et al. 1996. Extracellular matrix characterization during healing of full thickness wounds treated with a collagen/elastin dermal substitute shows improved skin regeneration in pigs. J Histochem Cytochem, 44, 1311-22.

78. L. Christensen, 1990. Fibronectin: a discrimination marker between small invasive carcinomas and benign proliferative lesions of the breast. Apmis, 98, 615-23.

79. M. V. Gulubova, 1996. Ultrastructural sinusoidal changes in extrahepatic cholestasis—light and electron microscopic immunohistochemical localization of collagen type Ill and type IV. Acta Histochem, 98, 271-83.

80. V. Everts, et al. 1994. Type VI collagen is phagocytosed by fibroblasts and digested in the lysosomal apparatus: involvement of collagenase, serine proteinases and lysosomal enzymes. Matrix Biol, 14, 665-76.

Some references concerning apoptosis include:

1A. A. Sarin, et al., Different interleukin-1 beta converting enzyme (ICE) family protease requirements for the death of T lymphocytes triggered by diverse stimuli. J Exp Med. Dec. 1, 1996; 184(6): 2445-50.

2A. P. Marchetti, et al., Mitochondrial permeability transition triggers lymphocyte apoptosis. J Immunol. Dec. 1, 1996; 157(11): 4830-6.

3A. K. Wang, et al., BID: a novel BH3 domain-only death agonist. Genes Dev. Nov. 15, 1996; 10(22): 2859-69.

4A. U. K. Mizoeva, et al., Inhibition of ICE-family cysteine proteases rescues murine lymphocytes from lipoxygenase induced apoptosis. FEBS Lett. Nov. 4, 1996; 396(2-3): 266-70.

5A. I. Rodriguez, et al., Systemic injection of a tripeptide inhibits the intracellular activation of CPP32-like protease and fully protects mice against Fas-mediated fulminant liver destruction and death. J Exp Med Nov. 1, 1996; 184(5): 206-72.

6A. E. M. Eves, et al., Apoptosis induced by differentiation or serum deprivation in an immortalized central nerve neuronal cell line. J Neurochem 1996 November; 67(5): 1908-20.

7A. J. Lotem, et al., Differential suppression by protease inhibitors and cytokines of apoptosis induced by wild and cytotoxic agents. Proc Natl Acad Sci USA. Oct. 29, 1996; 93(22): 12507-12.

8A. S. A. Susin, et al., Bcl-2 inhibits the mitochondrial release of an apoptogenic protease. J Exp Med. Oct. 1, 1996; 184(4): 1331-41,

9A. J. M. Glynn, et al., Apoptosis induced by HIV infection in H9 T cells is blocked by ICE-family protease inhibited by a Fas (CD95) antagonist. J Immunol. Oct. 1, 1996; 157(7): 2754-8.

10A. G. W. Meisenholder, et al., Events in apoptosis. Acidification is downstream of protease activation and BCL-2 protection. J Biol Chem. Jul. 5, 1996; 271(27): 16260-2.

11A. M. Muzio, et al., FLICE, a novel FADD-homologous ICE/CED-3-like protease, is recruited to the CD95 (FAS/APO-1) death-inducing signaling complex. F Cell. Jun. 14, 1996; 85(6): 817-27.

12A. M. D. Jacobson, et al., Role of Ced-3/ICE-family proteases in staurosporine-induced programmed cell death. J Cell Biol. 1996 June; 133(5): 1041-51.

13A. S. An, et al., Ligation of CD40 rescues Ramos-Burkitt lymphoma B cells from calcium ionophore- and receptor-triggered apoptosis by inhibiting activation of the cystein protease CPP32/Yama and cleavage of its substrate PARP. FEBS Lett. May 20, 1996; 386(2-3): 115-22.

14A. A. Yoshida, et al., Role of serine and ICE-like proteases in induction of apoptosis by etoposide in human leukemia cells. Leukemia. 1996 May; 10(5): 821-4.

15A. E. A. Slee, et al. Benzyloxycarbonyl-Val-Ala-Asp (OMe) fluoromethylketone (Z-VAD.FMK) inhibits apoptosis blocking the processing of CPP32. Biochem J. Apr. 1, 1996; 315 (Pt 1): 21-4.

16A. K. Cain, et al., A cleavage-site-directed inhibitor if interleukin-1 beta-converting enzyme-like proteases in apoptosis in primary cultures of rat hepatocytes. Biochem J. Feb. 15, 1996; 314 (Pt 1): 27-32.

17A. G. J. Pronk, et al., Requirement of an ICE-like protease for induction of apoptosis and ceramide generation by REAPER. Science. Feb. 9, 1996; 271(5250): 808-10.

Caspases are cysteine proteases which are involved in apoptosis or program cell death (PCD). They have a specific characteristics in that they cleave after the amino acid: aspartic acid. Several types of inhibitors were used against these enzymes such as: fluoromethyl ketones, aldehydes and chloromethyl ketones. One of the most successful inhibitors known for this family of cysteine proteases is the Z-VAD-FMK (Ref. 1A-17A) which is the property of Enzyme Systems Products, Inc., Livermore, Calif. The present invention describes a new generation of these inhibitors that extend their use to the therapeutic field.

Proteolysis is a key multistep process in the invasion of host tissue by cancer cells during tumor progression (Ref. 1 to 9). (The references are listed above.) Histopathological studies and in vitro studies of cultured cancer cells with metastatic potential have revealed that matrix metalloproteinases (Ref. 7,9,10,11), (Ref. 10) plasminogen activators (Ref. 7,12,13,14) and cathepsins (Ref. 11 and 15 to 24) are involved.

Sloane and co-workers earlier proposed that the presence of cathepsin B either at the plasma membrane of cancer cells or in the extracellular space around cancer cells is significant for metastasis (Ref. 11,15,16,17 and 25). Cathepsin B, the most prominent representative of the cysteine proteinase subclass (Ref. 26), is normally present in the lysosomes where it is involved in breakdown of proteins after phagocytosis or autophagy. When cathepsin B is blocked, lysosomal protein breakdown is significantly curtailed (Ref. 27,28). Under certain conditions, cathepsin B is not sorted to the lysosomes but secreted (Ref. 29,30,31), for example by macrophages during chronic inflammation (Ref. 32) and by chondrocytes during the acute phase of arthritis (Ref. 33,34). Secretion and association of cathepsin B with the plasma membrane have been found in metastatic cancer cells but not in cancer cells lacking this potential (Ref. 30,35,36). It is dependent on a functionally intact microtubular network (Ref. 30,31) and can be induced by acidification of the extracellular micro-environment (Ref. 30). Caspases are cysteine proteases which are involved in apoptosis or program cell death (PCD).

U.S. patents pertaining to the synthesis of amino acid moieties and synthesis of peptides are found in U.S. Pat. Nos. 3,531,258; 4,318,905; 5,527,882; and 5,847,695, which are incorporated by reference.

Other U.S. patents of interest include U.S. Pat. Nos. 4,518,528; 4,771,123; 5,416,013; 5,756,465; and 5,869,519 , all of which are incorporated herein by reference.

Prodrugs describe those usually nonactive compounds which are administered to a subject (i.e., a human) and are converted or cleaved in vivo to produce structures which have pharmacological and therapeutic properties.

All articles, patents, patent applications, standards, protocols, etc. cited in this application are incorporated herein by reference in their entirety.

As can be seen from the above description of the existing art, a need still remains for effective pro-drugs and protease inhibitors, particularly for the caspase enzymes. The present invention provides novel structures and pharmaceutical compositions and therapeutic agents which are useful as pro-drugs and as protease inhibitors. Methods of therapy using these structures and compositions are also claimed.

### SUMMARY OF THE INVENTION

The present invention concerns specific compounds generally described as having quinoline-(2-carbonyl)-(multiple-amino acids)-leaving group structures (and quinoline-type structures) which are useful as pro-drugs and as protease inhibitors particularly in caspase therapy for a wide range of disease conditions. Usually two, three or four amino acid linking groups are present.

In another aspect, the present invention concerns a compound of the structure:

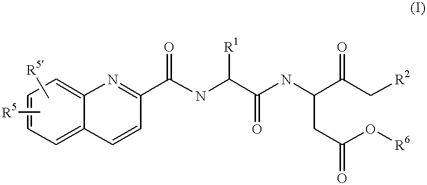

wherein in Structure I,

R¹ is selected from the group consisting of alkyl, substituted alkyl, aryl, and substituted aryl which group —N—CH—(R¹)—(C═O)— produces a natural amino acid structure or an unnatural amino acid structure, and;

the carbon adjacent to R¹ group is in the D or L configuration;

R² is selected from the group consisting of

—F;

and

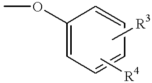

wherein R³ and R⁴ are each independently selected from the group consisting of hydrogen, alkyl, fluoro, chloro, carboxyl, alkoxy, alkyl carbonyl, aryl carbonyl, and amino;

and R⁵ and R^{5′} are each independently selected from hydrogen, alkyl, alkoxy, fluoro, chloro, carboxy, alkyl carbonyl, aryl carbonyl, amino, and together can form a cyclic ring or a heterocyclic ring; and

R⁶ is selected from alkyl having 1 to 10 carbon atoms, aryl or substituted aryl;

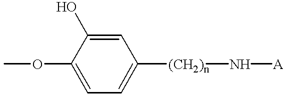

wherein A is a covalently bonded amine protecting group, and

n is 1-4, preferably 2;

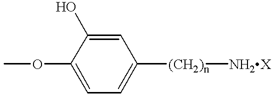

wherein X is a pharmaceutically acceptable salt, and n is 1-4, preferably 2; or

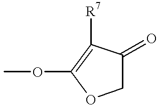

wherein R⁷ is selected from the group consisting of alkyl having 1 to 10 carbon atoms, aryl and alkylaryl.

In another aspect, the present invention concerns a pharmaceutical composition for use as a protease inhibitor having a compound selected from the structure:

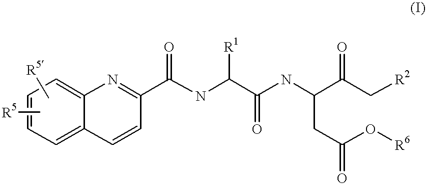

wherein in Structure I

R¹ is selected from the group consisting of alkyl, substituted alkyl, aryl, and substituted aryl which group —N—CH(R¹)—(C═O)— produces a natural amino acid structure or an unnatural amino acid structure, and;

the carbon adjacent to R¹ group is in the D or L configuration;

R² is selected from the group consisting of

—F;

and

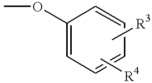

wherein R³ and R⁴ are each independently selected from the group consisting of hydrogen, alkyl, fluoro, chloro, carboxyl, alkoxy, alkyl carbonyl, aryl carbonyl, and amino;

and R⁵ and R^{5′} are each independently selected from hydrogen, alkyl, alkoxy, fluoro, chloro, carboxy, alkyl carbonyl, aryl carbonyl, amino and together can form a cyclic ring structure or a heterocyclic ring structure; and

R⁶ is selected from alkyl having 1 to 10 carbon atoms, aryl or substituted aryl;

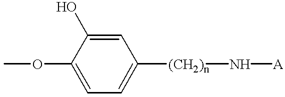

where A is a covalently bonded amine protecting group, and

n is 1-4, preferably 2; and

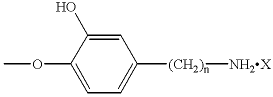

where X is the pharmaceutically accepted salt, and

n is 1-4, preferably 2; and

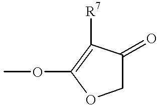

wherein R⁷ is selected from the group consisting of alkyl having 1 to 10 carbon atoms, aryl and alkylaryl or the

pharmaceutically acceptable acid or base salts thereof and a pharmaceutically accepted excipient.

In specific embodiments of the pharmaceutical composition, in the structure:

R¹ is selected from isopropyl or isobutyl;

R² is F ; and R⁵ is hydrogen.

In specific embodiments of the pharmaceutical composition, in the structure:

R¹ is selected from isopropyl or isobutyl;

R² is

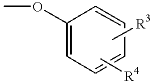

wherein R³ and R⁴ are each fluoro; and R⁵ is hydrogen, preferably when R³ and R⁴ are fluoro in the 2 and 6 positions of the phenyl ring.

In specific embodiments, R² is independently selected from

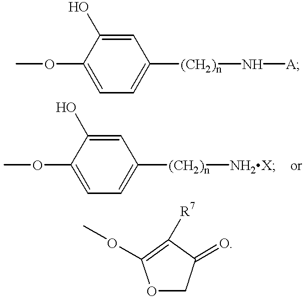

In another embodiment, the present invention pharmaceutical composition for use as a protease inhibitor of the structure:

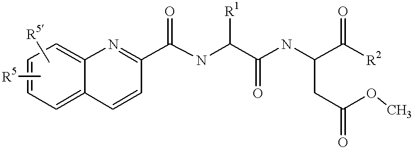

wherein

R¹ is selected from the group consisting of methyl, ethyl, isopropyl, and iso-butyl;

R² is selected from the group consisting of:

—F

or

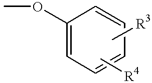

wherein R³ and R⁴ are each independently selected from the group consisting of hydrogen, alkyl having 1 to 10 carbon atoms, carboxyl, fluoro, chloro and amino;

and R⁵ and R⁵′ are each independently selected from the group consisting of hydrogen having 1 to 10 carbon atoms, alkyl having 1 to 10 carbon atoms, alkoxyl having 1 to 10 carbon atoms, fluoro, and chloro;

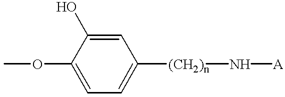

wherein A is a covalently bonded amine protecting group, and

n is 1-4, preferably 2;

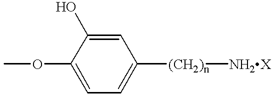

wherein X is a pharmaceutically acceptable salt, and or

n is 1-4, preferably 2; and

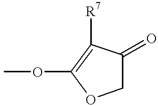

wherein R⁷ is selected from the group consisting of alkyl having 1 to 10 carbon atoms, aryl and alkylaryl.

In yet another embodiment, the present invention concerns a pharmaceutical composition for use as an inhibitor to caspase or a caspase—like enzyme having a structure selected from the group consisting of:

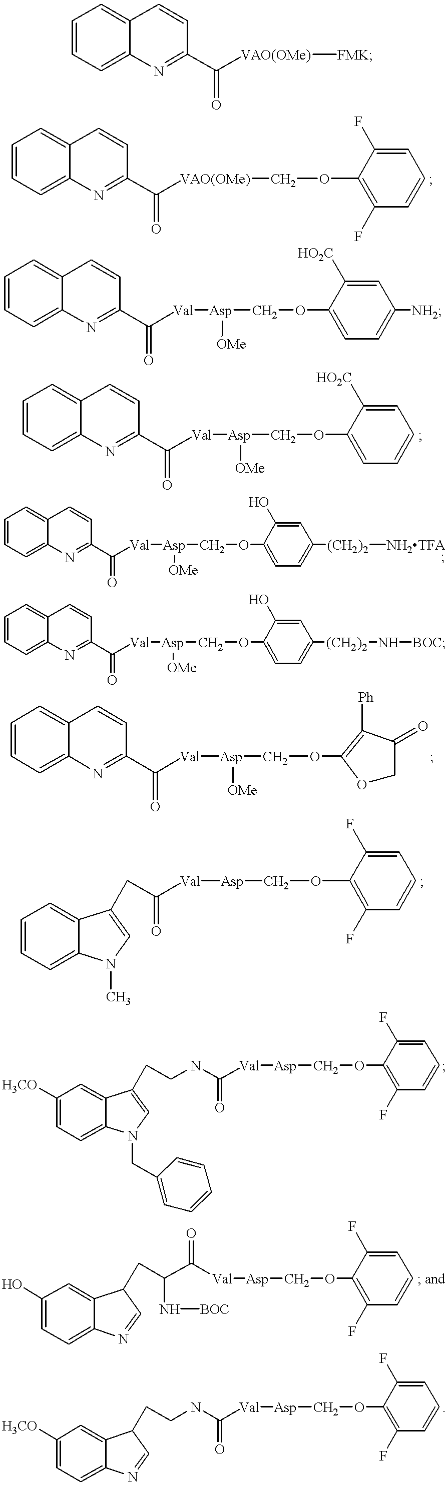

In still another embodiment, the present invention concerns a pharmaceutical composition comprising a compound of the structure:

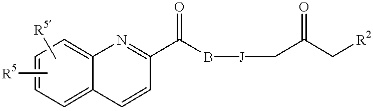

wherein B and J is each selected from groups creating a natural amino acid structure or an unnatural amino acid structure, and;

the amino acid is in the D or L configuration;

R² is selected from the group consisting of

—F

and

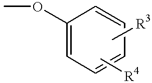

wherein R³ and R⁴ are each selected from the group consisting of hydrogen alkyl, fluoro, chloro, carboxyl, alkoxy, alkyl carbonyl, aryl carbonyl, and amino; and

R⁵ and R⁵′ are each independently selected from hydrogen, alkyl, alkoxy, fluoro, chloro, carboxy, alkoxy, alkyl carbonyl, aryl carbonyl, and amino.

In another aspect, the present invention concerns reagents (compounds) useful as caspase inhibitors and pharmaceutical compositiond for use as a protease inhibitor having a compound selected from the structure:

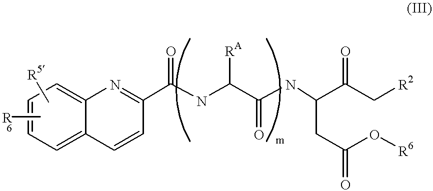

wherein in Structure III:

m is 1, 2 or 3, creating 1, 2 or 3 amino acid linkages, such that

when m=1, R^{A}=R¹,

when m=2, R^{A} is R¹ and R^{1B} in the separate amino acids and

when m=3, R^{A} is R¹, R^{1B} and R^{1C} wherein R¹, R^{1B} and R^{1C} in the separate amino acids which amino acids are the same or different amino acid when R¹, R^{1B} and R^{1C} are independently selected from the group consisting of alkyl, substituted alkyl, aryl, and substituted aryl which group —N—CH(R¹)—(C═O)—; N—CH(R¹)—(C═O)—NH—CH(R^{1B})—(C═O); or NCH(R¹)(C═O)—NH—CH(R^{1B})(C═O)—NHCH(R^{1C})(C═O)— produces natural amino acid structures or an unnatural amino acid structures, and;

the carbon adjacent to R¹ group is in the D or L configuration;

R² is selected from the group consisting of:

—F;

and

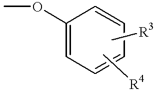

wherein R³ and R⁴ are each independently selected from the group consisting of hydrogen, alkyl, fluoro, chloro, carboxyl, alkoxy, alkyl carbonyl, aryl carbonyl, and amino;

and R⁵ and R^{5′} are each independently selected from hydrogen, alkyl, alkoxy, fluoro, chloro, carboxy, alkyl carbonyl, aryl carbonyl, amino and together form a cyclic ring structure or a heterocyclic ring structure; and

R⁶ is selected from alkyl having 1 to 10 carbon atoms, aryl or substituted aryl;

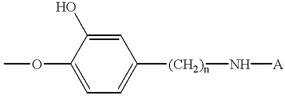

wherein A is a covalently bonded amine protecting group, and

n is 1-4, preferably 2;

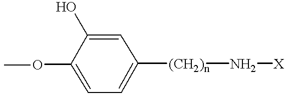

where X is the pharmaceutically accepted salt, and

n is 1-4, preferably 2; and

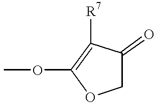

wherein R⁷ is selected from the group consisting of alkyl having 1 to 10 carbon atoms, aryl and alkylaryl or the pharmaceutically acceptable acid or base salts thereof, and a pharmaceutically acceptable excipient.

In specific embodiments when m=2, R¹ and R^{1B} are each independently selected from methyl, ethyl, isopropyl and t-butyl.

In specific embodiments when m=3, R¹, R^{1B} and R^{1C} are each independently selected from methyl, ethyl, isopropyl and t-butyl.

Others.

### BRIEF DESCRIPTION OF THE FIGURES

FIG. 1 shows a specific structure of the present invention having two amino acids and the fluoromethyl ketone moiety.

FIG. 1A is a specific structure of the present invention having three amino acids and included the fluoromethyl ketone moiety.

FIG. 2 shows a specific structure having the difluorophenoxy moiety.

FIG. 2A is a specific structure of the present invention having three amino acids and include the difluorophenoxy moiety.

FIG. 3 shows a specific structure having a 4-amino-2-carboxylic acid moiety.

FIG. 4 shows a specific structure having a 2-carboxylic acid moiety.

FIG. 5 shows a specific structure having a dopamine structure as a trifluoro acetic acid salt.

FIG. 6 shows a specific structure having a dopamine structure with a t-butoxy protecting group.

FIG. 7 shows a specific structure having a tetronic acid moiety.

FIGS. 8 to 29 illustrate the inhibitory effect of the novel compounds on various caspases. The activity for each compound is described as the concentration that reduces the maximum response by 50% (IC₅₀).

FIG. 8 is a graphic representation which illustrates the inhibitory effect of the quinoline-(2-carbonyl)-V-D(OMe)—CH₂-4-amino salicylic acid against caspase 9 showing the log of the concentration in μM versus % inhibition.

FIG. 9 is a graphic representation which illustrates the inhibitory effect of the quinoline-(2-carbonyl)-V-D(OMe)—CH₂-4-amino salicylic acid against caspase 8 showing the log of the concentration in μM versus % inhibition.

FIG. 10 is a graphic representation which illustrates the inhibitory effect of the quinoline-(2-carbonyl)-V-D(OMe)—CH₂-4-amino salicylic acid against caspase 1 showing the log of the concentration in μM versus % inhibition.

FIG. 11 is a graphic representation which illustrates the inhibitory effect of the quinoline-(2-carbonyl)-V-D(OMe)—CH₂-4-amino salicylic acid against caspase 3 showing the log of the concentration in μM versus % inhibition.

FIG. 12 is a graphic representation which illustrates the inhibitory effect of indole-3-V-D(OMe)—CH₂—O—Ph against caspase 1 showing the log of the concentration in μM versus % inhibition.

FIG. 13 is a graphic representation which illustrates the inhibitory effect of melatonin-V-D(OMe)—CH₂—O—Ph against caspase 1 showing the log of the concentration in μM versus % inhibition.

FIG. 14 is a graphic representation which illustrates the inhibitory effect of Bzl-melatonin-V-D(OMe)—CH₂—O—Ph with caspase 1 showing the log of the concentration in μM versus % inhibition.

FIG. 15 is a graphic representation which illustrates the inhibitory effect of hydroxy Trp-TTP-V-D(OMe)—CH₂—O—Ph with caspase 1 showing the log of the concentration in μM versus % inhibition.

FIG. 16 is a graphic representation which illustrates the inhibitory effect of TFA Trp-V-D(OMe)—CH₂—O—Ph TFA with caspase 1 showing the log of the concentration in μM versus % inhibition.

FIGS. 17A and 17B are graphic representations which illustrates the inhibitory effect of non-esterase treated (17 A) and esterase treated (17 B) quinoline-(2-carbonyl)-L-D(OMe)—CH₂—F (the FMK) against caspase 9 showing the log of the concentration in μM versus % inhibition.

FIGS. 18A and 18B are graphic representations which illustrates the inhibitory effect of non-esterase treated (18A) and esterase treated (18B) quinoline-(2-carbonyl)-V-D(OMe)—CH₂—F (the FMK) against caspase 9 showing the log of the concentration in μM versus % inhibition.

FIG. 19 is a graphic representation which illustrates the inhibitory effect of quinoline-(2-carbonyl)-V-D(OMe)—CH₂— salicylic acid against caspase 1 showing the log of the concentration in μM versus % inhibition.

FIG. 20 is a graphic representation which illustrates the inhibitory effect of quinoline-(2-carbonyl)-V-D(OMe)—CH₂—(4-amino salicylic acid against caspase 3 showing the log of the concentration in μM versus % inhibition.

FIG. 21 is a graphic representation which illustrates the inhibitory effect of quinoline-(2-carbonyl)-L-D-CH₂—(—OPh) against caspase 1 showing the log of the concentration in μM versus % inhibition.

FIG. 22 is a graphic representation which illustrates the inhibitory effect of hydroxy quinoline-(2-carbonyl)-V-D(—OMe)(—CH₂—OPh) against caspase 1 showing the log of the concentration in μM versus % inhibition.

FIG. 23 is a graphic representation which illustrates the inhibitory effect of esterase treated quinoline-(2-carbonyl)-L-D(OMe)—CH₂—F (the FMK) against caspase 1 showing the log of the concentration in μM versus % inhibition.

FIG. 24 is a graphic representation which illustrates the inhibitory effect of esterase treated quinoline-(2-carbonyl)-V-D(OMe)—CH₂—F (the FMK) against caspase 1 showing the log of the concentration in μM versus % inhibited.

FIGS. 25A and 25B are graphic representations which illustrates the inhibitory effect of non-esterase (25A) and esterase treated (25B) of quinoline-(2-carbonyl)-L-D(OMe)—CH₂—F (the FMK) against caspase 3 showing the log of the concentration in μM versus % inhibition.

FIG. 26 is a graphic representation which illustrates the inhibitory effect of quinoline-(2-carbonyl)-L-D-CH₂—OPh against caspase 1 showing the log of the concentration in μM versus % inhibition.

FIG. 27 is a graphic representation which illustrates the inhibitory effect of quinoline-(2-carbonyl)-V-D-CH₂—OPh against caspase 1 showing the log of the concentration in μM versus % inhibition.

FIG. 28 is a graphic representation which illustrates the inhibitory effect of quinoline-(2-carbonyl)-L-D-CH₂—OPh against caspase 3 showing the log of the concentration in μM versus % inhibition.

FIG. 29 is a schematic representation showing the structures of some natural amino acids.

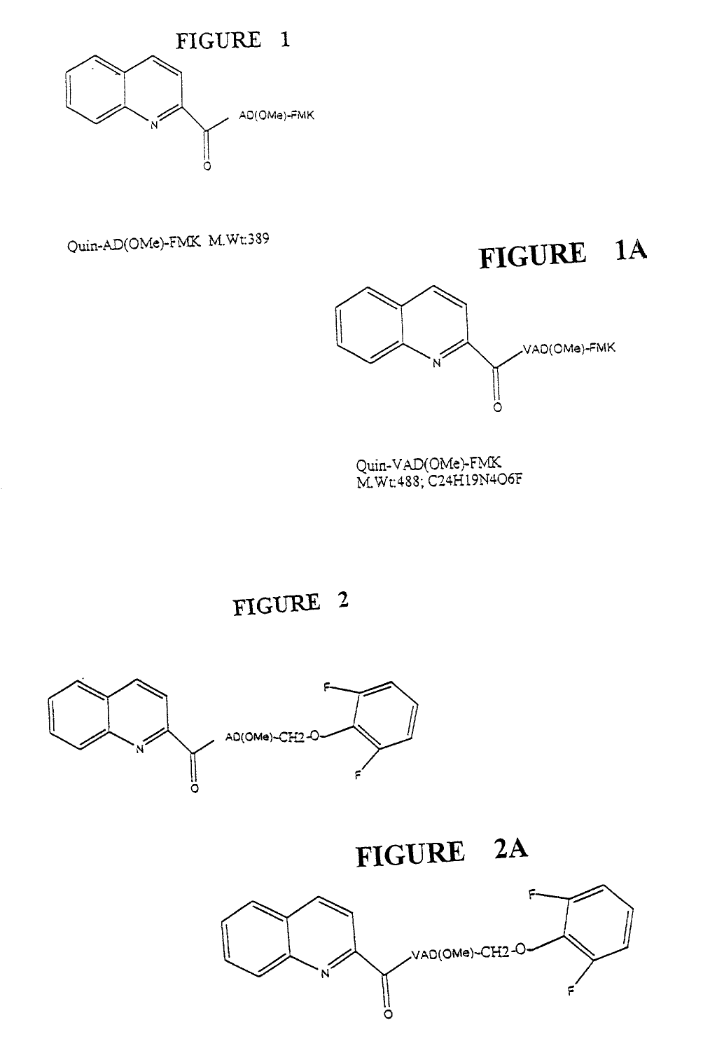

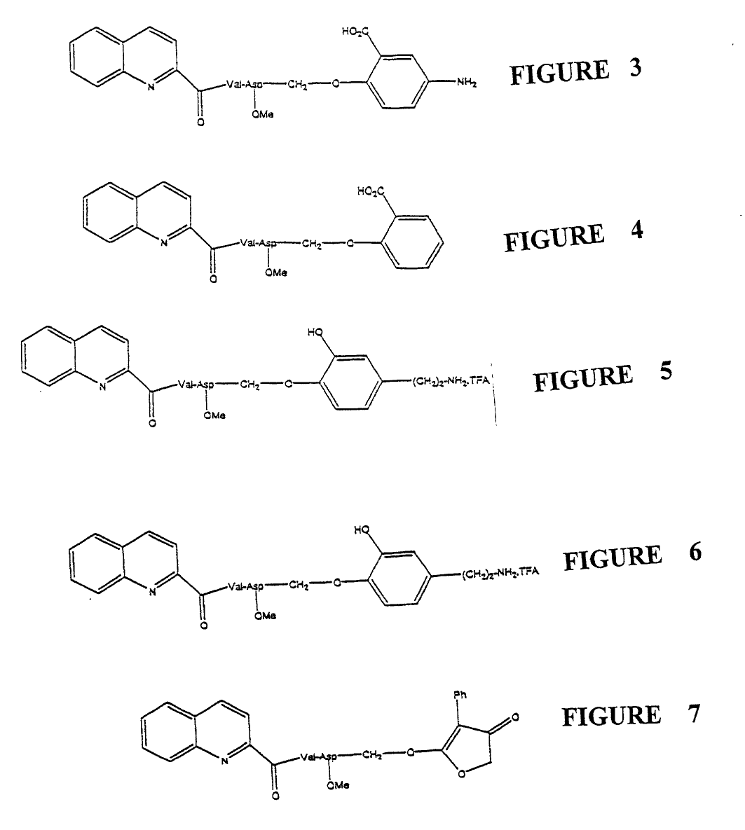

### DETAILED DESCRIPTION OF THE INVENTION AND PREFERRED EMBODIMENT

Definitions:

As used herein:

“Alkyl” refers to the alkyl groups having between about 1 and 20 carbon atoms and preferably between about 1 and 10 carbon atoms. All configurations of the alkyl groups are within the term “alkyl”. Methyl and ethyl are more preferred.

“Alkoxy” or “alkoxyl” refers to the common alkyl-o-moiety having between about 1 and 20 carbon atoms and preferably between about 1 and 10 carbon atoms. All configurations of these alkyl groups are within the terms “alkoxy” or “alkoxyl”. Methyl and ethyl are more preferred.

“Amino acid” refers to those organic compounds which include natural amino acids and synthetic (or unnatural) amino acids. The natural amino acids are the basis of all living systems having an amino group and a carbonyl group which are connected by a carbon which contains various substituents. The natural amino acids are all in the L-configuration as is shown in Table 1. The D-configuration amino acids are known but do not participate in metabolic processes. Synthetic amino acids are any other amino acids than the natural amino acids found in Table 1 below and in FIG. 27.

TABLE 1
CONVENTIONAL AMINO ACID DESIGNATIONS

| | ONE-LETTER | THREE-LETTER |
|---|---|---|
| AMINO ACID | SYMBOL | SYMBOL |
| alanine | A | ala |
| arginine | R | arg |
| asparagine | N | asn |
| aspartic acid | D | asp |
| cysteine | C | cys |
| glutamic acid | E | glu |
| glutamine | Q | gln |
| glycine | G | gly |
| histidine | H | his |
| isoleucine | I | ile |
| leucine | L | leu |
| lysine | K | lys |
| methionine | M | met |
| phenylalanine | F | phe |
| proline | P | pro |
| serine | S | ser |
| threonine | T | thr |
| tryptophan | W | trp |
| tryosine | Y | tyr |
| valine | V | val |

The terms “natural and unnatural amino acid” refers to both the naturally occurring amino acids and other non-proteinogenic α-amino acids commonly utilized by those in the peptide chemistry arts when preparing synthetic analogues of naturally occurring peptides, including D and L forms. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, y-carboxylglutamic acid, arginine, ornithine and lysine. Examples of unnatural alpha-amino acids include hydroxylysinc, citrulline, kynurenine, (4-aminophenyl) alanine, 3-(2′-naphthyl)alanine, 3-(1′-naphtyl)alanine, methionine sulfone, (t-butyl)alanine, (t-butyl)glycine, 4-hydroxyphenyl-glycine, aminoalanine, phenylglycine, vinylalanine, propargyl-glycine, aminoalanine, phenylglycine, vinylalanine, propargyl-glycine, 1,2,4-triazolo-3-alanine, thyronine, 6-hydroxytryptophan, 5-hydroxytryptophan, 3-hydroxy-kynurenine, 3-aminotyrosine, trifuloromethylalanine, 2-thienylalanine, (2-(4-pyridyl)ethyl) cysteine, 3,4-dimethoxy-phenylalanine, 3-(2′-thiazolyl)alanine, ibotenic acid, 1-amino-1-cyclopentane-carboxylic acid, 1-amino-1-cyclohexanecarboxylic acid, quisqualic acid, 3-(trifluoromethylphenyl)alanine, (cyclohexyl)glycine, thiohistidine, 3-methoxytyrosine, norleucine, norvaline, alloisoleucine, homoarginine, thioproline, dehydro-proline, hydroxyproline, homoproline, indoline-2-carboxylic acid, 1,2,3,4-tetrahydroisoquinoline-3-carboxylic acid, 1,2,3,4-tetrahydroquinoline-2-carboxylic acid, α-amino-n-butyric acid, cyclohexylalanine, 2-amino-3-phenylbutric acid, phenylalanine substituted at the ortho, meta, or para position of the phenyl moiety with one or two of the following groups: a (C₁to C₄) alkyl, a (C₁ to C₄) alkoxy, a halogen or a nitro group, or substituted once with methylenedioxy group; β-2- and 3-thienylalanine; β-2- and 3-furanylalanine; P-2-, 3- and 4-pyridylalanine; β-(benzothienyl-2- and 3-yl)alanine; β-(1- and 2-naphthyl)alanine; O-alkylated derivatives of serine, threonine or tyrosine; S-alkylated cysteine, S-alkylated homocysteine, the O-sulfate, O-phosphate and O-carboxylate esters of tyrosine; 3-(sulfo) tyrosine, 3-(carboxy)tyrosine, 3-(phospho)tyrosine, the 4-methanephosphonic acid ester of tyrosine, 3,5-diiodotyrosine, 3-nitrotyrosine, e-alkyllysine, and delta-alkyl ornithine. Any of these α-amino acids may be substituted with a methyl group at the alpha position, a halogen at any position of the aromatic residue on the α-amino side chain, or an appropriate protective group at the O, N, or S atoms of the side chain residues. Appropriate protective groups are discussed above.

“Amino-protecting group” refers to substituents of the amino group commonly employed to block or protect the amino functionality while reacting other functional groups of the molecule. The term “protected (monosubstituted) amino” means there is an amino-protecting group on the monosubstituted amino nitrogen atom. Examples of such amino-protecting groups include the formyl (“For”) group, the trityl group, the phthalimido group, the trichloroacetyl group, the trifuloroacetyl group, the chloroacetyl, bromoacetyl, and iodoacetyl groups, urethane-type protecting groups, such as t-butoxycarbonyl (“Boc”), 2-(4-biphenylyl)propyl-2-oxycarbonyl (“Bpoc”), 2-pheylpropyl-2-oxycarbonyl (“poc”), 2-(4-xenyl) isopropoxycabonyl, 1,1-diphenylethyl-1-oxycarbonyl, 1,1-diphenypropyl-1-oxycarbonyl, 2-(3,5-dimethoxyphenyl) propyl-2-oxycarbonyl (“Dd”), 2-H-toluyl)propyl-2-oxycarbonyl, cyclopentanyloxycarbonyl, 1-methylcyclopentanyloxycarbonyl, cyclohexanyloxycarbonyl, 1-methyl-cyclohexanyloxycarbonyl-carbonyl, 1-methyl-cyclohexanyloxycaarbonyl, 2-methylcyclohexanyloxycarbonyl, 2-(4-toluysulfonyl)ethoxycarbonyl, 2-(methylsulfonyl) ethoxycarbonyl, 2-(triphenylphosphino)ethoxycarbonyl, 9-fluorenylmethoxycarbonyl (“Fmoc”), 2-(trimethylsilyl)ethoxycarbonyl, allyloxycarbonyl, 1-(trimethylsilylmethyl)prop-1-enyloxycarbonyl, 5-benzisoxalymethoxycarbonyl, 4-acetoxybenzyl-oxycarbonyl, 2,2,2-trichloroethoxycarbonyl, 2-ethynyl-2-propoxycarbonyl, cyclopropylmethoxycarbonyl, isobomyloxycarbonyl 1-piperidyloxycarbonyl, benzyloxycarbonyl (“Cbz”), 4-phenylbenzyloxycrbonyl, 2-methylbenzyloxycarbonyl, α-2,4,5,-tetramethylbenzyloxycarbonyl (“Tmz”), 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, 4-(decyloxy)benzyloxycarbonyl and the like; the benzoyhnethylsulfonyl group, the 2,2,5,7,8-pentamethylchroman-6-sulfonyl group (“PMC”), the dithiasuccinoyl (“Dts”) group, the 2-(nitro) phenyl-sulfonyl group (“Nps”), the diphenylphosphine oxide group, and like amino-protecting groups. The species of amino-protecting group employed is not critical so long as the derivatized amino groups is stable to the conditions of the subsequent reaction(s) and can be removed at the appropriate point without disrupting the remainder of the molecule. Preferred amino-protecting groups are Boc, Cbz and Fmoc.

“Leaving group” refers to those leaving groups conventional in the art. Preferred leaving groups include fluoromethyl ketone, 2,6-difluorophenoxy, 2-carboxyphenoxyl, 2-carboxy-4-amino-phenoxyl, tetronic acid and the like.

“Quinoline” refers to the standard 1-aza-naphthalene structure. The term “quinoline” also includes those “quinoline-type” structures wherein the 2- or 3- position has a carbonyl moiety, e.g., from quinic acid.

“Quinoline-type” also refers to the standard indole structure and the indole structure wherein the 2- or 3- position has a carbonyl moiety. Quinoline-type also refers to melatonin and substituted melatonin structures.

“Substituted alkyl group” refers to an alkyl (as defined herein) wherein a proton has been replaced with a chloro or fluoro or any group which found in a natural or unnatural amino acid.

“Aryl” refers to phenyl, napthalyl and the like.

“Substituted aryl” refers to those mono or di-substituted phenyl or naphthyl found in the art. The substitutents include alkyl (as defined herein), alkoxyl (as defined herein), fluoro, chlro, carboyxl (wherein the alkyl or aryl is as defined herein), alkyl carbonyl (wherein the alkyl is as defined herein), aryl carbonyl (wherein aryl is as defined herein) or amino.

“Structure designations” for general and for specific structures (see Table 2 (see A¹ to J¹ below)) refer to Q-V-D(OCH₃)—CH₂—FMK described the quinoline-(C═O)-valinyl aspartic acid wherein the valinyl is connected to the quinic acid through the amine and to the aspartic acid amine through the carbonyl group. In some structures, Val is replaced by Leu, one carboxylic acid of the aspartic acid is protected as the methyl ester and the other carboxyl group has been altered. That hydroxyl group of —C(═O)—OH has been converted to a methylene group (See Examples). In one embodiment, the methylene group is terminated by a fluorine (—F) creating a terminal fluoromethyl ketone moiety (FMK) or in another embodiment is terminated by an unsubstituted to substituted phenoxy group (—O—) e.g. 2,6-difluoro. Other leaving groups are also used in place of the phenoxy group.

TABLE 2
STRUCTURE DESIGNATIONS

| STRUCTURE | |
|---|---|
| DESIGNATION | STRUCTURE |
| A¹ | Q-βAla-Asp(OMe)-FMK |
| | (where Q is 2-quinoline —C═O) |
| B¹ | 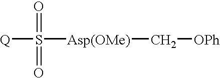 |
| C¹ | Q-βAla-Asp(OMe)—CH₂—OPh |
| D¹ | Q-Ala-Asp(OMe)-FMK* |
| E¹ | HQ-Ala-Asp(OMe)—CH₂—OPh |
| F¹ | MQ-Ala-Asp(OMe)—CH₂—OPh |
| G¹ | 2-Val-Ala-Asp(OMe)—CH₂—OPh |
| H¹ | 4HQ-Ala-Asp(OMe)—CH₂—OPh |
| I¹ | Q-Val-Asp(OMe)—CH₂—OPh*** |
| J¹ | Q-Leu-Asp(OMe)—CH₂—OPh** |

FMK is fluoromethyl ketone —C(═O)CH₂F
—OPh is 2,6-difluorophenoxy
***Best result
**Second best result
*Third best result

Discussion

Cysteine proteases are important enzymes in the biological system. As the name indicates, they contain the amino acid cysteine in the active sites of these enzymes. They are known to be tissue-degrading enzymes that manifest themselves in several disease states. The cathepsins belong to the cysteine proteases with about 20 individual enzymes within this family. Some of the diseases that involve cathepsins are arthritis, metastases and multiple sclerosis. Additional diseases for example include: infectious diseases, such as meningitis and salpingitis, septic shock, respiratory diseases; inflammatory conditions, such as arthritis, cholangitis, colitis, encephalitis, endocerolitis, hepatitis, pancreatitis and reperfusion injury, ischemic diseases such as the myocardial infarction, stroke and ischemic kidney disease; immune-based diseases, such as hypersensitivity; auto-immune diseases, such as multiple sclerosis; bone diseases; and certain neurodegenerative diseases. The caspases are another type of cysteine protease enzymes. They are involved in the main cascade that is known to be the main cause behind apoptosis or program cell death (PCD).

This invention presents a unique group of novel peptide caspase inhibitors. Their composition and preliminary activities show a great promise as potential pharmaceuticals.

These structures are viewed as covalently bonded moieties

A′—B′—C′

which consistuent groups are defined:

A′ is the unsubstituted or substituted quinoline or quinoline-type structure;

B′ comprises two, three or four natural D- or L-amino acids or unnatural amino acids e.g. including those which may also have the L-configuration. More preferably these amino acids are selected from glutamic acid, valine, aspartic acid or a monoalkyl (i.e. methyl) protected aspartic acid. Most preferably the amino acids in the structure II is valine-aspartic acid (O—Me).

C′ is a leaving group. These leaving groups are generally defined in the Summary above and in the claims. Preferably these leaving groups include, but are not limited, fluoromethylketone; 2,6-difluorophenoxy; 4-amino-2-carboxy phenoxy; 2-carboxy phenoxy; L-dopamine-trifluoroacetic acid (DOPA·TFA); L-dopamine-t-butoxycarbonyl (DPOA-BOC); tetronic acid; melatonin; and the like. In addition, group C′ may also have, when released in vivo in the body, its own useful pharmaceutical actions.

The literature shows that the caspase inhibitors are needed as useful therapeutics in several disease states, i.e. as Alzheimer's, Amyltrophic Lateral Sclerosis (ALS), Huntington's disease, meningitis, spinal chord injuries and liver damage. It is known that control of apoptosis may have utility in treating disease (see Rodriguez, Ref.5A). Specifically, inhibitors of the ICE/CED-3 family may have therapeutic effects. For example, it has been suggested that inhibition of ICE may be useful in the treatment of inflammatory disorders (Dolle, et al., J. Med. Chem., 37:563, 1994; Thomberry, et al., Biochemistry, 33:394, 1994). It is also known that inhibitors of ICE/CED-3 family members may have utility in treating degenerative diseases such as neurodegenerative diseases (e.g., Alzheimer's disease, Parkinson's disease, amyotrophic lateral sclerosis (ALS), Huntington's disease), ischemic disease of heart or central nervous system (i.e., myocardial infarction and stroke), and traumatic brain injury, as well as in alopecia, AIDS and toxin induced liver disease (Nicholson, Nature Biotechnology 14:297, 1996). They also represent a very important role in cells and tissue preservation.

Apoptosis is an exhaustively studied field at the present time with its ultimate therapeutic potential is still beyond the horizon. These inhibitors present themselves as very important new therapeutic reagents for a variety of disease conditions.

### Utility and Administration

The compounds of the invention have been shown to effect reduced programmed cell death in various in vitro animal preparations and tissue cultures, and accordingly are useful in the affecting physiological phenomena. These compounds have been shown to be effective in animal models and are, therefore, useful in treating a mammal, particularly a human being.

These compounds are also useful as immunosuppressants, and in particular they are useful in the treatment of autoimmune diseases, such as arthritis, etc.

Administration of the active compounds and salts described herein can be via any of the accepted modes of administration for therapeutic agents which affect apoptosis and other conditions created by traumatic premature cell death. These methods include oral, parenteral, transdermal, subcutaneous and other systemic modes. The preferred method of administration is oral, except in those cases where the subject is unable to ingest, by himself, any medication. In those instances it may be necessary to administer the composition parenterally.

Depending on the intended mode, the compositions may be in the form of solid, semi-solid or liquid dosage forms, such as, for example, tablets, suppositories, pills, capsules, powders, liquids, suspensions, skin patch, or the like, preferably in unit dosage forms suitable for single administration of precise dosages. The compositions will include a conventional pharmaceutical excipient and an active compound of formula I or the pharmaceutically acceptable salts thereof and, in addition, may include other medicinal agents, pharmaceutical agents, carriers, adjuvants, diluents, etc.

The amount of active compound administered will, of course, be dependent on the subject being treated, the severity of the affliction, the manner of administration and the judgement of the prescribing physician. However, an effective dosage is in the range of 0.1-100 mg/kg/day, preferably 0.5-5 mg/kg/day. For an average 70 kg human, this would amount to 7-7000 mg per day, or preferably 35-350 mg/day. Alternatively, the administration of compounds as described by L. C. Fritz et al. in U.S. Pat. No. 6,200,969 is followed. One of skill in the art with this disclosure can create an effective pharmaceutical formulation.

Since the effects of the compounds herein are achieved through the same central mechanism (effecting apoptosis in the living system) dosages (and forms of administration) are within the same general and preferred ranges for all these utilities.

For solid compositions, conventional non-toxic solid include, for example, pharmaceutical grades of mannitol, lactose, starch, magnesium stearate, sodium saccharin, talcum, cellulose, glucose, sucrose, magnesium carbonate, and the like may be used. The active compound as defined above may be formulated as suppositories using, for example, polyalkylene glycols, for example, propylene glycol, as the carrier. Liquid pharmaceutically administrable compositions can, for example, be prepared by dissolving, dispersing, etc. an active compound as defined above and optional pharmaceutical adjuvants in a excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington 's Pharmaceutical Sciences, Mack Publishing Company, Easton, Pa., 17^{th} Edition, 1985. The composition or formulation to be administered will, in any event, contain a quantity of the active compound(s), a therapeutically effective amount, i.e. in an amount effective to alleviate the symptoms of the subject being treated.

For oral administration, a pharmaceutically acceptable non-toxic composition is formed by the incorporation of any of the normally employed excipients, such as, for example pharmaceutical grades of mannnitol, lactose, starch, magnesium stearate, sodium saccharin, talcum, cellulose, glucose, sucrose, magnesium, carbonate, and the like. Such compositions take the form of solutions, suspensions, tablets, pills, capsules, powders, sustained release formulations and the like. Such compositions may contain 10%-95% active ingredient, preferably 1-70%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, etc.

A more recently devised approach for parenteral administration employs the implantation or skin patch for a slow-release or sustained-release system, such that a constant level of dosage is maintained. See. e.g., U.S. Pat. No. 3,710,795, which is incorporated herein by reference.

The following preparations and examples serve to illustrate the invention. They should not be construed as narrowing it, or limiting its scope in any way.

Experimental

The starting material compounds, solvents, reagents, etc. described herein are available from commercial sources or are easily prepared from literature references by one of skill in the art. See Chem Sources USA, published annually by Directories Publications, Inc. of Boca Raton, Fla. Also see The Aldrich Chemical Company Catalogue, Milwaukee, Wis. The starting materials are used as obtained unless otherwise noted.

### EXAMPLE 1

### Synthesis of Boc-Asp(OMe)—CHN₂

Boc-Asp(OMe)—OH (5.0 g, 20.2 mmol.) was dissolved in anhydrous tetrahydrofuran THF (50 ml). After cooling to −15° C. (ice-salt bath), 4-methyl morpholine (2.8 ml, 26.3 mmol) was added followed by isobutyl chloroformate (2.8 ml, 22.3 mmol) dropwise. The reaction was stirred for 15 min. The precipitate was filtered. Diazomethane made freshly from 10.0 g of DIAZALD was added at −10° C. and stirred for one hour. The solution was warmed to room temperature and stirred for 4 hours. The solvent was removed. The residue diazomethane was purified by silica gel column chromatograph (Eluting with 10% to 30% EtOAc in hexanes). Yield: 5.2 g (94.9% yield). δ_{H}(300 MHz, CDCl₃) 5.67 (broad 1H), 4.52 (broad, 1H), 3.69 (s, 3H), 3.03 (m, 1H), 2.70 (M, 1H), 1.45 (s, 9H).

### EXAMPLE 2

### Synthesis of Boc-Asp(OMe)-α-(2-oxy-2,6-Difluorophenyl)

Boc-Asp(OMe)—CHN₂ (1.12 g, 4.13 mmol.) was dissolved in THF: Ether (1:1 30 ml) and cooled to −15° C. HBr/acetic acid (30%, 0.98 ml, 4.96 mmol) in ether: THF (1:1, 8 ml) was added dropwise and stirred for 15 minutes. Thin layer chromatography (TLC) showed complete reaction. Brine (50 ml) was added. The water layer was extracted with THF: Ether (1:1, 50 ml). The organic layer was washed with aqueous NaHCO₃ (50 ml) and saturated NaCl (50 ml and dried over MgSO₄. The solvent was removed and pumped dry. Yield: 1.2 g (90%). This bromide (1.2 g, 3.7 mmol) was dissolved in dimethylformamide DMF (7 ml). 2,6-Difluorophenol (529 mg, 4.07 mmol) was added followed by KF (537 mg, 9.25 mmol) and stirred overnight. EtOAc (100 ml) was added. The EtOAc solution was washed with water (50 ml), aqueous NaHCO₃ (50 ml), and saturated NaCl (50 ml) and dried over MgSO₄. The solvent was removed. The residue was purified by column chromatograph on silica gel (mesh size 230-400) (eluent: 10% to 30% EtOAc in hexanes). Yield: 1.1 g (80%). δ_{H}(300 MHz, CDCl₃) 6.95 (m, 3H), 5.04 (s, 24), 4.73 (broad, 1H), 3.10 (m, 1H), 2.85 (M, 2H), 1.45 (s, 9H).

### EXAMPLE 3

### Synthesis of Quinoline-(2-Carbonyl)-Valine-OH

Quinic acid (quinoline-2-carboxylic acid) (2.0 g, 11.5 mmol), Val-O-t-Bu, HCl (2.42 g, 11.5 mmol), HOBT (1.56 g, 11.5 mmol), and HBTU (4.38 g, 11.5 mmol) were dissolved in DMF (15 ml). Diisopropyl ehtylamine (6 ml, 34.6 mmol) was added using a syringe and stirred for 1 hr. EtOAc (100 ml) was added. The EtOAc solution was washed with water (100 ml), aqueous NaHCO₃ (100 ml), saturated NaCl (100 ml) and dried over MgSO₄. The solvent was removed. The residue was purified by column chromatograph on silica gel (mesh size 230-400) (eluting with 50% EtOAc in hexanes). Yield: 3.5 g (92.3% yield). The tert-butyl ester (3.5 g, 10.6 mmol) was dissolved in 95% trifluoroacetic acid (TFA) (35 ml), and stirred for 1 hr. The solution was stripped down, chased with addition of hexanes (3×5 ml) and pumped dry. Yield: 2.8 g (96% yield). MS(E1): M⁺=273.

### EXAMPLE 4

### Synthesis of Quinoline-2-(C═O)-Val-Asp(OMe)-α-(2-oxy-2,6-Difluorophenyl) Methyl Ketone

Boc-Asp(Ome)CH₂-2-(2-oxy-2,6-difluorophenyl) methyl ketone (150 mg, 0.40 mmol), was dissolved in 95% TFA (3ml) and stirred for one hour. The solution was stripped under vacuum, chased with hexanes (3×5 ml) and pumped dry. Q-(C═O)-Val-OH (110mg, 0.40 mmol), HOBT (55 mg, 0.40 mmol), HBTU (153 mg, 0.040 mmol) were added to the residue obtained in DMF (3 ml). DIEA (0.21 ml, 1.2 mmol) was added using a syringe and stirred for 1 hr.

EtOAc (60 ml) was added and the EtOAc solution was washed with H₂O (50 ml), aqueous NaHCO₃ (50 ml), saturated NaCl (50 ml) and dried over MgSO₄. The solvent was removed. The residue was purified by two preparatives TLC (20×20) plate (Developing with 50% EtOAc in hexanes. Yield: 90 mg (43% yield). δ_{H}(300 MHz, 8.75 (t, 1H), 8.32 (m, 1H), 8.29 (m, 1H0, 8.25 (d, 1H), 7.88 (d, 1H), 7.79 (t, 1H), 7.64 (t, 1H), 7.37 (t, 1H), 6.87 (t, 1H), 6.75 (t, 1H), 5.08 (M, 2H), 4.59 (m, 1H), 3.61 (s, 3H), 3.14 (m, 1H), 3.09 (m, 1H), 2.41 (m, 1H), 1.09 (m, 1H), MS (E.1), MH⁺=528.

### EXAMPLE 5

### Synthesis of Quinoline β A-D (OMe)-α-Fluoromethyl Ketone

Synthesis of 2-Quinoline(C═O)-Val-Asp(OMe)—OH—

Quinoline-(C═O)-val-OH (1.17 g, 4.29 mmol), Asp(OMe)—OBz, HCl (1.175 g, 4.29 mmol), HOBT (580 mg, 4.29 mmol), HBTU (1.63 g, 4.29 mmol) were dissolved in DMF (7 ml). DIEA (2.2 ml, 12.9 mmol) was added and stirred for 1 hr. EtOAc (100 ml) was added. The EtOAC and aqueous layers were separated and the EtOAc fraction was washed with water, NaHCO₃ solution, NaCl solution, and dried over MgSO₄. The EtOAc was removed. The residue was purified by column chromatography on silica gel (eluting with 50% EtOAc in hexanes). Yield: 0.7 g (67%).

The benzyl ester (1.4 g) was dissolved in EtOAc (100 ml). 10% Palladium on carbon (140 mg) was added and the solution was hydrogenated at 190 psi overnight. The solution was filtered through CELITE. The solvent was removed to give the acid. Yield: 1.0 g (87%). Analyzed by Mass Spec & NMR: MS(EI): MH⁺=402. δ_{H}(300 MHz, CDCl₃) 8.89 (d, 1 H), 8.31 (d, 1H), 8.27 (d, 1H), 8.17 (d, 1H), 7.87 (d, 1H), 7.78 (m, 1H), 7.65 (t, 1H), 7.44 (d, 1H), (m, 1H), 4.68 (m, 1H), 3.05 (m, 2H), 2.34 (m, 1H), 1.08 (m, 6H).

To Boc-D-(OMe)-FMK (527 mg, 0.002 mol) was added 95% trifluoroacetic acid (10 ml). The reaction mixture was stirred for 30 min. at ambient conditions and evaporated to dryness (at reduced pressure) to give the trifluoroacetic acid salt. To the salt was added dimethylformamide (10 ml) followed by quinoline-(C═O)-β-A-OH (500 mg, 0.002 mol), HOBT (276 mg, 0.002 mol), HBTU (775 mg, 0.002 mol) and DIEA (1.1 ml, 0.0063 mol). The reaction mixture was stirred for 30 min., extracted with EtOAc, which was washed with 10% hydrochloric acid, water, saturated NaHCO₃, and water, and dried over anhydrous MgSO₄, and evaporated (under reduced pressure). The crude product was purified by column chromatography—silica gel (230-400 mesh) by elution with 95:5/ethyl acetate:methanol give product 110 mg (14% yield). The structure was confirmed by mass spectral and nuclear magnetic response spectroscopy by a commercial analytical laboratory.

### EXAMPLE 6

### Synthesis of 2-Quinoline-(C═O)-A-D (OMe)-α-Fluoromethyl Ketone Synthesis of 2-Quinoline-(C═O)-Val-Asp(OMe)—CH₂Br

Quinoline-(C═O)-val-asp(OMe)—OH (2.06 g, 5.14 mmol) was dissolved in THF (60 ml) and cooled to −15° C. NMM (0.73 ml, 6.68 mmol) was added followed by IBCF (0.73 ml, 5.65 mmol) and stirred for 0.5 hr. The precipitate was filtered off. Diazomethane made from 5.0 g of diazald was added at −10° C. and stirred for 1 hr. The solution was warmed to ambient temperature and stirred for 4 more hr. The solvent was removed. The diazoketone was purified by column chromatography on silica gel (eluting with 50% EtOAc in hexanes). Yield: 1.5 g (69%).

The diazoketone (415 mg, 0.98 mmol) was dissolved in THF: ether (1:1, 30 ml) and cooled to −15° C. HBr/acetic acid (30%, 0.24 ml, 1.17 mmol) in THF: ether (1:1, 6 ml) was added dropwise. TLC showed the complete reaction in about 20 min. Brine (NaCl) was added. The aqueous layer was extracted with THF: ether (1:1, 50 ml). The EtOAC and aqueous layers were separated and the EtOAc fraction was washed with water, NaHCO₃ solution, NaCl solution, and dried over MgSO₄ , and concentrated to dryness. (Pumped dry.) Yield: 450 mg (96%). MS(EI): MH⁺=479.

To Boc-aspartic acid (OMe) fluoromethylketone (1.6 g, 0.006 mol) was added 95% trifluoroacetic acid (25 ml). The reaction mixture was stirred for 30 min. (at ambient conditions) and evaporated to dryness (under reduced pressure) to produce the trifluoroacetic acid salt. To the solution of TFA salt in dimethylformamide (25 ml) was added quinoline alanine (1.48 g, 0.006 mol), HOBT (840 mg, 0.0062 mol), HBTU (2.4 g, 0.006 mol) and DIEA (3.2ml, 0.18 mol). The reaction mixture was stirred for 1 hr (at ambient temperature), and extracted with ethyl acetate (3×100 mol). The ethyl acetate extract was washed with 10% aqueous hydrochloric acid (1×100 ml), saturated NaHCO₃(1×100 ml), water (1×100 ml), and the extract was evaporated under reduced pressure to give a crude product which was purified by column chromatography (silica gel, 230-400 mesh). Elution was with 70:30/ethylacetate:hexane gave pure fractions which were combined and evaporated under reduced pressure to produce the desired product—yield 850 mg (36% yield). The structure was confirmed by mass spectral and nuclear magnetic resonance spectral analysis of samples analyzed by a commercial analytical laboratory.

### EXAMPLE 7

### Synthesis of Quinoline-(2-Carbonyl)-V-D-(OMe)-α-CH₂F (the Fluoromethyl Ketone)

To Boc-D-CH₂-I (OMe) (426 mg, 0.0016 mol) was added 95% trifluoroacetic acid (15 ml). The reaction mixture was stirred for 30 min. (at ambient conditions) and evaporated to dryness (under reduced pressure) to give the trifluoroacetic acid salt. To the solution of trifluoroacetic acid salt in dimethylformamide (10 ml) was added quinoline-(2-carbonyl)-V—OH (450 mg, 0.0016 mol), HOBT (230 mg, 0.0017 mol), HBTU (650 mg, 0.0017 mol) and DIEA (850 microliter, 0.0048 mol). The reaction mixture was stirred for 30 min. at ambient conditions and extracted with ethyl acetate (3×100 ml). The ethyl acetate extract was washed with 10% aqueous hydrochloric acid (1×100 ml), saturated NaHCO₃ solution (1×100 ml) water (1×100 ml), and dried over anhydrous MgSO₄. The extract was separated and evaporated (under reduced pressure) to give a crude product which was purified by column chromatography over silica gel, (230-400 mesh). Elution used 80:20/ethylacetate:hexane and gave a pure product—125 mg (18.5% yield). The structure was confirmed by mass spectral and nuclear magnetic resonance spectral analysis by a commercial analytical firm.

### EXAMPLE 8

### Synthesis of Quinoline-(2-Carbonyl)-L-D-(OMe-α-CH₂F (the Fluoromethyl Ketone) TFA

To Boc-D (OMe)—CH₂F (the fluoromethyl ketone) (340 mg, 0.0013 mol) was added 95% trifluroacetic acid (15 ml). The reaction mixture was stirred for 30 min. (at ambient conditions) and evaporated to dryness (at reduced pressure) to give the TFA salt. To the solution of the TFA salt in dimethylformamide, (10 ml) was added quinoline-(2-carbonyl)-L-OH (385 mg, 0.0013 mol), HOBT (190 mg, 0.0014 mol), HBTU (535 mg, 0.0014 mol) and DIEA (700 microliter). The reaction mixture was stirred for 30 min. (at ambient conditions) and extracted with EtOAc (300 ml). The EtOAc extract was washed with 10% HCl, saturated NaHCO₃ water and dried over anhydrous MgSO₄. The solvent was removed (by reduced pressure) to give crude product which was purified by column chromatography over silica gel (230-400 mesh). Elution was with 60:40/EtOAc:hexane to gave pure product 200 mg (35% yield). The structure was confirmed by mass spectral analysis and nuclear magnetic resonance spectroscopy by a commercial analytical firm.

### EXAMPLE 9

As described herein, similar related compounds and compositions can be produced by one of skill in the art by following the steps described in Examples 1-8.

By replacement of the recited amino acids (or protected amino acids) in the described process steps, a variety of quinoline-(2-carbonyl)-amino acid-amino acid-CH₂F (the fluoromethyl ketone) or quinoline-(2-carbonyl)-amino acid-amino acid-phenoxy moiety is obtained.

### EXAMPLE 10

### Experimental Protocol for Caspase Inhibitors (Tunel Analysis)

In this assay, cells are pretreated with caspase inhibitors and subjected to an apoptotic induction by exposure to Actinomycin D. The TUNEL assay demonstrates the DNA fragmentation resulting from the induction of the apoptotic cascade. Flow Cytometry measures the percent of the cell population undergoing apoptosis. The effect of the inhibitors is identified as a reduction in the percent of the cell population undergoing apoptosis.

Cell Type: WEHI 231 cells, murine immature B cells (suspension) Plate cells at 2×10⁵/ml in 10 ml of media (Total 2×10⁶ cells) Preincubate caspase inhibitors (Formula I) one hour before inducing apoptosis.

Caspase Inhibitor Stock Solution is 20 mM in 100% DMSO.

Actinomycin D (ActD) stock is 1 μg/μl add 10 μl/10 ml)-Treat 4 h

Process for Tunel Analysis. (See Table 2 below).

TABLE 2

| | | | | | | | | | | | | |
|---|---|---|---|---|---|---|---|---|---|---|---|---|
| PROCESS FOR TUNEL ANALYSIS+HZ,1/64 | | | | | | | | | | | | |
| | 0 | DMSO | 20 μM* | 100 μM* | ActD* | 1 μM* + | 10 μM* + | 20 μM* + | 30 μM* + | 40 μM* + | 50 μM* + | 100 μM* + |
| | - | 0.5% | alone | alone | - − | ActD | ActD | ActD | ActD | ActD | ActD | ActD |
| Volume | - | 50 μl | 10 μl | 50 μl | — | 0.5 μl | 5 μl | 10 μl | 15 μl | 20 μl | 25 μl | 50 μl |
| of Stock | | | | | | | | | | | | |

*Inhibitor level

Cell Processing for Tunel Analysis and Flow Cytometry.

Centrifuge cells at 300× g for 5 min and aspirate supernatant.

Resuspend cells in 5.0 ml of cold 1× PBS, Spin at 300× g, 5 min.

Aspirate supernatant. Add 5 ml of cold 1% paraformaldehyde in 1× PBS. Incubate for 15 min. on ice. Spin at 300× g for 5 min. Aspirate supernatant. Wash cells with 5.0 ml of cold 1× PBS. Spin at 300× g for 5 min.

Aspirate supernatant. Wash cells with 5.0 ml of cold 1× PBS.

Spin at 300× g for 5 min. Aspirate supernatant.

Resuspend the cell in 300 μl of cold IX PBS, Transfer to a 1.5 ml tube. Add dropwise—while medium vortexing—700 μl of cold absolute ethanol.

Store at −20° C. at least 18 hr prior to Tunel Labeling

Fixed cells are stable for at least 30 days.

Process fixed cells for Tunel using the Apo-BRDU kit from Pharmingen (San Diego, Calif.) following the manufacturer's instructions and analyze by flow cytometry.

TABLE 3
CONCENTRATIONS

| Compound | | | | | | | |
|---|---|---|---|---|---|---|---|
| Tested | 1 μM/Act | 10 μM/Act | 20 μM/Act | 30 μM/Act | 40 μM/Act | 50 μM/Act | 100 μM/Act |
| Z-VAD-FMK | 33% | 64% | 17% | 19% | 12% | 8% | 6% |
| A¹ | 62% | 57% | 42% | 40% | 15% | 32% | 9% |
| B¹ | 58% | 74% | 75% | 53% | 70% | 70% | 53% |
| C¹ | 88% | 87% | 72% | 71% | 78% | 68% | 3% |
| D¹* | 88% | 67% | 16% | 2% | 1% | 2% | 1% |
| E¹ | 29% | 38% | 13% | 25% | 7% | 12% | 10% |
| F¹ | 32% | 35% | 47% | 61% | 36% | 20% | 21% |
| G¹ | 56% | 62% | 57% | 52% | 62% | 56% | 8% |
| H¹ | 81% | 92% | 88% | 91% | 85% | 82% | 83% |
| I¹*** | 88% | 2% | 3% | 2% | 3% | 2% | 2% |
| J¹** | 88% | 16% | 3% | 2% | 2% | 2% | 3% |

Values represent % non-viable cells. A lower percentage value indicates a greater inhibitory activity.
***Best result
**Second best result
*Third best result
For ester A¹ to J¹ designations, see page 29 above.

Table 4 below demonstrates the lack of toxcity of dimethylsulfoxide (DMSO) and inhibitors at the concentrations used in this assay.

TABLE 4
CONCENTRATIONS**

| Compound | | | | | |
|---|---|---|---|---|---|
| Tested | Untreated | DMSO | 20 μM Inh | 100 μM Inh | ACT |
| Z-VAD-FMK | 7% | ND | 3% | 7% | 71% |
| A¹ | 5% | ND | 5% | 5% | 63% |
| B¹ | 4% | ND | 2% | 4% | 51% |
| C¹ | 3% | ND | 3% | 4% | 82% |
| D¹ | 3% | 39% | 6% | 8% | 92% |
| E¹ | 1% | 1% | 1% | 1% | 39% |
| F¹ | 6% | 0% | 5% | 5% | 35% |
| G¹ | 2% | 2% | 1% | 1% | 65% |
| H¹ | 2% | 2% | 3% | 2% | 90% |
| I¹ | 2% | 2% | 2% | 53% | 91% |
| J¹ | 2% | 1% | 1% | 1% | 91% |

**For ester designations A¹ to J¹ see page 29 above.

### EXAMPLE 10A

### Synthesis of t-Butyl 5-N-Boc-Salicylate

5-Amino-salicylic acid (2.5 g, 16.3 mmol) was dissolved in a mixture of dioxane (25 ml), water (10 ml), and NaOH (653 mg, 16.3 mmol) in 15 ml of water. The solution was stirred and cooled in an ice water bath. (Boc)₂O (3.92 g, 18.0 mmol) was added and stirring was continued at ambient temperature for 1 hr. The solution was concentrated to about 15 ml using reduced pressure, cooled in an ice water bath, covered with a layer of ethyl acetate (50 ml), and acidified with a dilute solution of KHSO₄ to pH 2-3. The aqueous phase was extracted with EtOAc (50 ml). The EtOAc extracts were washed with water (2×50 ml), NaCl solution, and dried over MgSO₄, concentrated to give the n-Boc salicylic acid. Yield: 3.9 g (95%). Nmr: δ_{H} (300 MHz, CDCl₃) 13.8 (broad, 1H), 9.26 (s, 1H), 7.49 (dd, 1H), 6.85 (d, 1H), 1.5 (s, 9H).

A solution of n-Boc salicylic acid (1.85 g, 7.3 mmol) in DMF (20 ml) cooled at 0° C. was treated with 1,1′-carbonyldiimidazole (1.42 g, 8.8 mmol). After 1 hr at ambient temperature, t-butyl alcohol (1.4 ml, 14.6 mmol) and (DBU) (1.31 ml, 8.8 mmol) were added, stirred for 2 hr, and poured into cooled water (50 ml). The aqueous layer was extracted with EtOAc (100 ml). The EtOAc and aqueous layers were separated and the EtOAc fraction was washed with NaCl solution and dried over MgSO₄. The solvent was removed. The residue was purified by column chromatograph on silica gel (eluting with 60% EtOAc in hexanes). Yield: 1.79 g (79%). It was analyzed by nmr: δ_{H} (300 MHz, CDCl₃) 7.65 (m, 1H), 7.49 (broad, 1H), 7.26 (s, 1H), 6.90 (d, 1H), 1.60 (s, 9H), 1.54 (s, 9H).

### EXAMPLE 11A

### Synthesis of Quinoline-(2-Carbonyl)-Val-Asp(OMe)—OH

Quinoline-(2-carbonyl)-val-OH (1.17 g, 4.29 mmol), Asp(OMe)—OBz.HCl (1.175 g, 4.29 mmol), HOBT (580 mg, 4.29 mmol), HBTU (4.63 g, 4.29 mmol) were dissolved in DMF (7 ml). DIEA (2.2 ml, 12.9 mmol) was added and stirred for 1 hr. EtOAc (100 mol) was added. The EtOAc fraction was separated, washed with water, NaHCO₃ and NaCl, and dried over MgSO₄. The solvent was removed. The residue was purified by column chromatograph on silica gel (eluting with 50% EtOAc in hexanes). Yield: 0.7 g (67%). The benzyl ester (1.4 g) was dissolved in EtOAc (100 mol). 10% Palladium on carbon (140 mg) was added. The solution was hydrogenated at 190 psi overnight (20 hr) and filtered through CELITE. The solvent was removed to produce the acid. Yield: 1.0 g (87%) MS(EI): MH⁺=402. _{H}(300 MHz, CDCl₃) 8.89 (d, 1H). 8.31 (d, 1H), 8.27 (d, 1H), 8.17 (d, 1H), 7.87 (d, 1H), 7.78 (m, 1H), 7.65 (t, 1H), 7.44 (d, 1H), (m, IH), 4.68 (m, 1H), 3.05 (m, 2H), 2.34 (m, 1H), 1.08 (m, 6H).

### EXAMPLE 11B

### Synthesis of Quinoline-(2-Carbonyl)-Val-Asp(OMe)—CH₂Br

Quinoline-(2-Carbonyl)-val-asp(OMe)—OH (2.06 g, 5.14 mmol) was dissolved in THF (60 ml). Cooled to −15° C. NMM (0.73 ml, 6.68 mmol) was added followed by IBCF (0.73 ml, 5.65 mmol). The reaction mixture was stirred for 0.5 hr and the precipitate was filtered off. Diazomethane made from 5.0 g of DIAZALD was added at −10° C., stirred for 1 hr., warmed to room temperature, and stirred for 4 more hr. The solvent was removed. The diazoketone was purified by column chormatograph on silica gel (eluting with 50% EtOAc in hexanes). Yield: 1.5 g (69%). TLC: R_{f}(ethyl acetate: hexane=1:1)=0.35. The diazoketone (415 mg, 0.98 mmol) was dissolved in THF:ether (1:1.30 ml), cooled to −15° C. HBr/acetic acid (30%, 0.24 ml, 1.17 mmol) in THF:ether (1:1, 50 ml) was added and extracted. The organic fraction was separated and washed with water, NaHCO₃, NaCl, and dried over MgSO₄. The product was concentrated to dryness and pumped dry. Yield: 450 mg (98%). TLC: Rf (ethyl acetate: hexane=1:1)=0.45. MS(EI): MH⁺=479.

### EXAMPLE 11C

### Synthesis of Quinoline-(2-Carbonyl)-Val-Asp(OMe)-α-(2-oxy-Amino Salicylic Acid)

Quinoline-(2-Carbonyl)-val-asp(OMe)—CH₂Br (250 mg, 0.52 mmol) was dissolved in DMF (5 ml). t-Butyl n-Boc salicylate (102 mg, 0.52 mmol) was added followed by KF (76 mg, 1.3 mmol) and stirred overnight. EtOAC (50 ml) was added. The EtOAC and aqueous layers were separated and the EtOAc fraction was washed with water, NaHCO₃ solution, NaCl solution, and dried over MgSO₄. The residue was purified by preparative TLC plate (eluting 50% EtOAc in hexanes). Yield: 190 mg (62%). The butyl ester was (45 mg, 0.076 mmol) was dissolved in 95% TFA (2 ml), stirred for 1 hr, stripped down and chased with hexane (3×3 ml). After pumping dry, the yield was 35 mmg (86%). MS(EI): MH⁺=551.

### EXAMPLE 12

### Synthesis of t-Butyl Salicylate

Salicylic acid (1.5 g, 10.9 mmol) in dimethylformamide (DMF) (20 ml) cooled at ° C. was treated with 1,1′-carbonyldiimidazole (2.11 g, 13.0 mmol). After 1 hr at ambient temperature, t-butyl alcohol (2.1 ml, 21.8 mmol) and (DBU) (1.95 ml, 13.0 mmol) were added. The solution was stirred for 2 hr and poured in cooled water (50 ml), extracted with EtOAc (100 ml). The EtOAc and aqueous layers were separated and the EtOAc fraction was washed with NaCl solution and dried over MgSO₄. The solvent was removed. The residue was purified by column chromatography on silica gel (eluting with 30% EtOAc in hexanes). Yield: 1.5 g (71%). It was analyzed by nmr: δ_{H}(300 MHz, CDCl₃) 7.77 (dd, 1H), 7.40 (m, 1H), 6.95 (dd, 1H), 6.84 (m, 1H), 1.56 (s, 9H).

### EXAMPLE 13

### Synthesis of Quinoline-(2-Carbonyl)-Val-Asp(OMe)—CH—O-Salicylic Acid

Quinoline-(2-carbonyl)-val-asp(OMe)—CH₂Br (250 mg, 0.52 mmol) was dissolved in DMF (5 ml). t-Butyl salicylate (102 mg, 0.52 mmol) was added followed by KF (76 mg, 1.3 mmol) and stirred overnight (about 2 hr). EtOAc (50 ml) was added. The EtOAC and aqueous layers were separated and the EtOAc fraction was washed with water, NaHCO₃ solution, NaCl solution, and dried over MgSO₄. The residue was purified by preparative TLC plate (eluting 50% EtOAc in hexanes). Yield: 190 mg (62%). MS(EI): MH⁺=592. The butyl ester was (45 mg, 0.076 mmol) was dissolved in 95% TFA (1 ml), stirred for 1 hr, stripped down and chased with hexane (3×3 ml), and then pumped dry. Yield: 35 mg (86%). MS(EI): MH⁺=536.

### EXAMPLE 14

### Synthesis of N-BOC Dopamine

Dopamine (2.5 g, 13.2 mmol) was dissolved in a mixture of dioxane (25 ml), water (10 ml), and NaOH (527 mg, 13.2 mmol) in 15 ml of water, then stirred and cooled in an ice water bath. (Boc)₂O (3.17 g, 14.5 mmol) was added and stirring was continued at ambient temperature for 1 hr. The solution was concentrated to about 15 ml, cooled in an ice water bath, covered with a layer of ethyl acetate (50 ml), and acidified with a dilute solution of KHSO₄ to pH 2-3. The aqueous phase was extracted with EtOAc (50 ml). The EtOAc and aqueous layers were separated and the EtOAc fraction was washed with water (2×50 ml), sodium chloride solution, and dried over MgSO₄ Solvent was removed. The residue was purified by column chromatography on silica gel (eluting with 50% EtOAc in hexanes). Yield: 2.6 g (78%). NMR: δ_{H}(300 MHz, CDCl₃) 6.79 (d, 1H), 6.70 (s, 1H), 6.59 (d, 1H), 4.60 (broad, 1H), 3.31 (t, 2H), 2.66 (t, 2H), 1.44 9s, 9H).

### EXAMPLE 15

### Synthesis of Quinoline-(2-Carbonyl)-Val-Asp(OMe)-α-(3-oxy-Dopamine)

Quinoline-(2-Carbonyl)-val-asp(OMe)—CH₂Br (250 mg, 0.52 mmol) was dissolved in DMF (5 ml). N-Boc-dopamine (132 mg, 0.52 mmol) was added followed by KF (76 mg, 1.3 mmol) and stirred overnight. EtOAc (50 ml) was added. The EtOAC and aqueous layers were separated and the EtOAc fraction was washed with water, NaHCO₃ solution, NaCl solution, and dried over MgSO₄. The residue was purified by preparative TLC plate (eluting 50% EtOAc in hexanes). Yield: 200 mg (60%). MS(EI): MH⁺=651. The butyl ester was (150 mg, 0.23 mmol) was dissolved in 95% Trifluoroacetic acid (TFA) (2 ml), stirred for 1 hr, stripped down and chased with hexane (3×3 ml), and pumped dry. Yield was 150 mg (98%). MS(EI): MH⁺=574.

### EXAMPLE 16

### Synthesis of Precursors of Tetronic Acid Preparation of Tetronic Acids: General Procedure

Solutions of lithium di-isopropylamide (LDA) were prepared at −78° C. under nitrogen by addition of solution of n-BuLi (1 equiv.) in hexane to a solution of di-isopropylamine (1.05 equiv.) in THF (ca. 1 mmol/ml). This solution was held at −78° C. for 25 min before generation of the lithium enolates of esters by addition of the appropriate ester (1 equiv.) in THF. The enolates were held at −78° C. for 25 min before addition of the required dioxolanone in THF. The reaction mixture was allowed to attain ambient temperature gradually (overnight stirring—about 20 hr) before evaporation of the solvent and partition of the residue between ether and water. The ethereal layer was washed with water. Acidification of the combined aqueous layers to about pH 1 with concentrated HCl gave the tetronic acid which was isolated by filtration or extraction.

### EXAMPLE 17A

### 2-(4-Methoxyphenyl) Tetronic Acid

By the general procedure, the dioxolanone (1.0 g, 6.4 mmol) was treated with the lithium enolate of methyl 4-methoxyphenylacetate (2.54 ml, 16.0 mmol). Acidification gave the crude product which was filtered off, dried, and recrystallized (ethyl acetate and hexane) to give the purified acid. Yield: 640 mg (48.5%). Analyzed by nmr: δ_{H}(300 MHz, DMSO-d₆) 7.84 (m, 2H), 6.95 (m, 2H), 4.74 (s, 2H), 3.75 (s, 3H).

### EXAMPLE 17B

### 2-(4-Fluoromethylphenyl)-Tetronic Acid

By the general procedure, the dioxolane (1.0 g, 6.4 mmol) was treated with the lithium enolate of methyl 4-fluorophenylacetate (2.69 g, 16.0 mmol). Acidification gave the crude product which was filtered off, dried, and recrystallised (ethyl acetate and hexane) to give the purified acid. Yield: 690 mg (56.0%). Analyzed by nmr: δ_{H}(300 MHz, DMSO-d₆) 7.96 (m, 2H), 7.23 (m, 2H), 4.77 (s, 2H).

### EXAMPLE 17C

### 2-(4-Trifluoromethylphenyl)-Tetronic Acid

By the general procedure, the dioxolane (1.0 g, 6.4 mmol) was treated with the lithium enolate of methyl 4-trifluoro-p-tolylacetate (3.49 g, 16.0 mmol). Acidification gave the crude product which was filtered off, dried, and recrystallised (ethyl acetate and hexane) to give the purified acid. Yield: 680 mg (43.5%). Analyzed by nmr: δ^{H}(300 MHz, DMSO-d₆) 8.17 (d, 2H), 7.73 (d, 2H), 4.81 (s, 2H).

### EXAMPLE 18

### Synthesis of Quinoline-(2-Carbonyl)-Val-Asp(OMe)—CH₂-2-Phenyltetronic Acid

Quinoline-(2-Carbonyl)-val-asp(OMe)—CH₂Br (210 mg, 0.44 mmol) was dissolved in DMF (5 ml). 2-Phenyltetronic acid (78 mg, 0.44 mmol) was added followed by KF (64 mg, 1.1 mmol) and stirred overnight. EtOAc (50 ml) was added. The EtOAC and aqueous layers were separated and the EtOAc fraction was washed with water, NaHCO₃ solution, NaCl solution, and dried over MgSO₄. The residue was purified by preparative TLC plate (eluting 50% EtOAc in hexanes). Yield: 125 mg (50%). MS(EI): MH⁺=574.

### EXAMPLE 19

### Synthesis of 5-Methoxytratamine-Carbonyl-Val-OH

A solution of 5-methoxytryptamine (500 mg, 2.63 mmol), in dry DMF (10 ml) cooled to 0° C. was treated with 1,1′-carbonyldiimidazole (426 mg, 2.63 mmol). After 1 hr at ambient temperature, Val-O-t-Bu.HCl (551 mg, 2.63 mmol), DBU (0.39 ml, 2.63 mmol), triethylamine (0.366 ml, 2.63 mmol) were added, and stirred overnight. EtOAc (80 ml) was added. The ethyl acetate layer was separated and washed with 1 N HCl, water, NaHCO₃, NaCl and dried over MgSO₄. The solvent was removed. The residue was purified by column chromatograph on silica gel (eluting with 50% EtOAc in hexanes). Yield: 0.7 g (68%). TLC: R_{f}(ethyl acetate: hexane=1:1)=0.40. MS(EI): MH⁺390.

t-Butyl ester (640 mg, 1.64 mmol) was dissolved in 95% TFA (7 ml). It was stirred for 1 hr, stripped down, chased with hexanes (3×5 ml) and pumped dry. Yield: 540 mg (98%), MS(EI): MH⁺⁼334.

### EXAMPLE 20

### Synthesis of 5-methoxytrytamine-carbonyl-Val Asp(OMe)-α-(2-oxy-2,6-Difluorophenyl) Methyl Ketone

Boc-asp (OMe)-α-(2-oxy-2,6-difluorophenyl) methyl ketone (345 mg, 0.93 mmol) was dissolved in 95% TFA (4 ml), stirred for 1 hr, stripped down, chased with hexanes (3×5 ml), and pumped dry. To this solution in DMF (7 ml) were added 5-methoxytrytamine-carbonyl-val-OH (308 mg, 0.93 mmol), HOBT (125 mg, 0.93 mmol), HBTU (351 mg, 0.93 mmol), followed by DIEA (0.48 ml, 2.8 mmol). The solution was stirred for 1 hr. EtOAc (50 ml) was added. The EtOAc layer was separated, washed with water, NaHCO₃, NaCl and dried over MgSO₄. The solvent was removed. The residue was purified by column chromatograph on silica gel (eluting with 75% EtOAc in hexanes). Yield: 0.7 g (44%), TLC: R_{f}(ethyl acetate: hexane=1:1)=0.15. MS(EI): MH⁺=589.

### EXAMPLE 21

### Evaluation Of Novel Compounds for IC₅₀ with Caspase 1, Caspase 3, Caspase 8 and Caspase 9

The determination of the IC₅₀/μm was performed according to Gary Johnson, 9401 James Avenue, Suite No. 155, Bloomington, Minn. 55431.

Caspase Inhibition Assay:—Caspases are diluted in caspase buffer, 0.1 M HEPES, 10% sucrose 0.1% CHAPS and 10 mM DTT, pH 7.5. Caspases 1, 3, and 8 are used at a concentration of 144 U/well and caspase 9 is used at 4.8 U/well in a 96 well fluorometric plate.

Inhibitors are initially dissolved in DMSO at 10 mg/ml and further dilutions can be prepared in caspase buffer. The inhibitors are generally tested at concentrations ranging from 50 uM to 0.005 uM. These are usually prepared as 2 fold or 2.5 fold decreasing dilutions. The enzyme approximately 144 U in 120 ul caspase buffer is added to 380 ul caspase buffer containing the appropriate concentration on the inhibitor and incubated on ice for 15 min. The reactants 200 ul are then added to a black fluorimetric plate and incubated in the fluorometer at 37° C. for 30 min.

During the incubation period appropriate coumarin substrates are prepared by dilution in DMSO and caspase buffer to provide a 0.417 mM working stock. AcYVAD-AFC is used for caspase 1, AcDEVD-AFC for caspase 3. AcIETD-AFC for caspase 8 and Ac-LEHD-AFC for capsase 9. Twenty five microliters of the stock solution are added to 200 ul of the caspase and inhibitor test solutions to give a final substrate concentration of 0.046 mM.

The fluorometer is set to 400 nm excitation and 505 nm emission. The enzyme-inhibitor-substrate is allowed to incubate at for an additional twenty min. and the response is read as fluorescence units vs inhibitor concentration. The responses are plotted as percent of the maximum response (response in the absence of inhibitor) for each concentration of the inhibitor. The inhibitory activity for each inhibitor is described as the concentration of inhibitor that produces a 50% inhibition of the maximum response (IC₅₀).

Some results are summarized below in Table 5.

TABLE 5
Q-VD(OMe)-W
(w is shown below)

| COMPOUND | CASP-1 | CASP-3 | CASP-8 |
|---|---|---|---|
| w | IC₅₀ | IC₅₀ | IC₅₀ |
| Q-VD(OMe)-ASA | 1.5 | 1.24 | 5.88 |
| Q-VD(OMe)-SA | 1.43 | 0.5 | |
| Q-VD(OMe)-DOPA- | | <25 | |
| BOC | | | |
| Q-VD(OMe)-Tetronic Acid | | 0.25 | |
| Q-VD(OMe)-DOPA.TFA | | 2 | |

*W is the leaving group

From this table, it can be seen that these compounds are specific protease inhibitors.

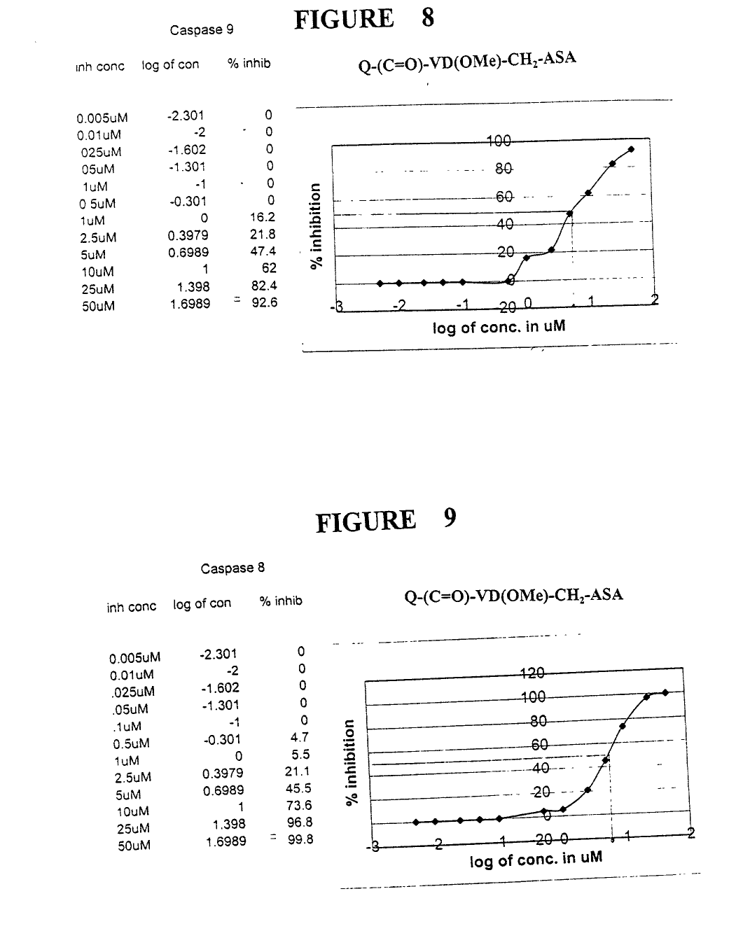

FIGS. 8 to 28 illustrate the inhibitor effect of the novel compounds with various caspases. The activity for each compound is described as the concentration that reduces the maximum response by 50% (IC₅₀).

FIG. 8 is a graphic representation which illustrates the inhibitory effect of the quinoline-(2-carbonyl)-V-D(OMe)—CH₂-4-amino-salicylic acid against caspase 9 showing the log of the concentration in μM versus % inhibition. The antilog of 0.735 is 5.44. The IC₅₀ is approximately 5.44 μM.

FIG. 9 is a graphic representation of the quinoline-(2-carbonyl)-V-D(OMe)—CH₂-4-amino salicylic acid with caspase 8 showing the log of the concentration in μM versus % inhibition. The IC₅₀ is approximately 5.82 μM.

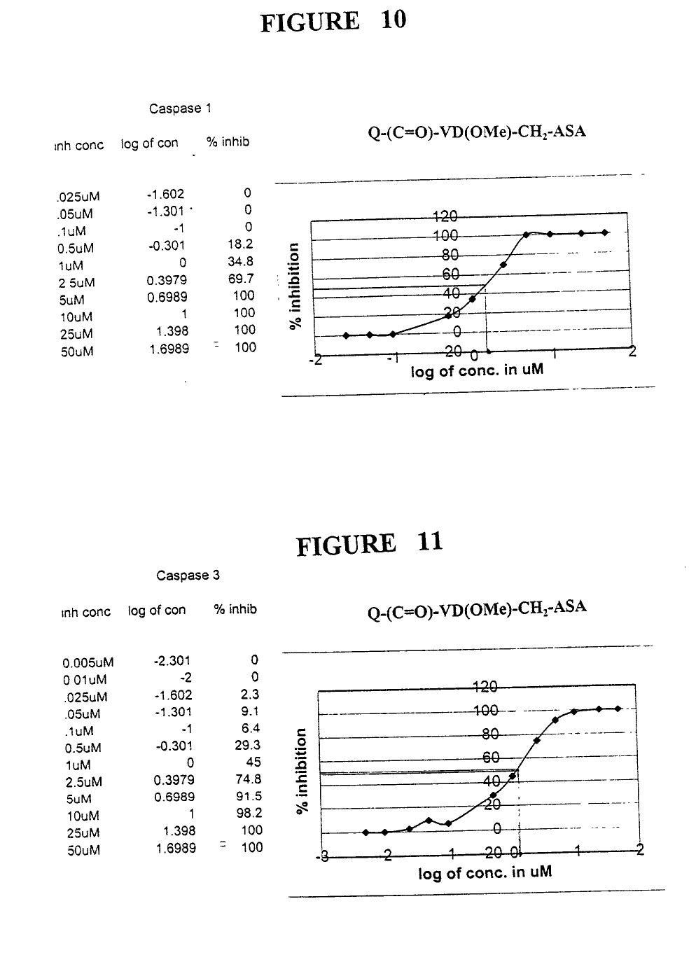

FIG. 10 is a graphic representation of the quinoline-(2-carbonyl)-V-D(OMe)—CH₂-4-amino salicylic acid with caspase 1 showing the log of the concentration in μM versus % inhibition. The antilog of 0.1636 is 1.46. The IC₅₀ is approximately 1.46 μM.

FIG. 11 is a graphic representation of the quinotine-(2-carbonyl)-V-D(OMe)—CH₂-4-amino salicylic acid with caspase 3 showing the log of the concentration in μM versus % inhibition. The antilog of 0.088 is 1.23. The IC₅₀ is approximately 1.23 μM.

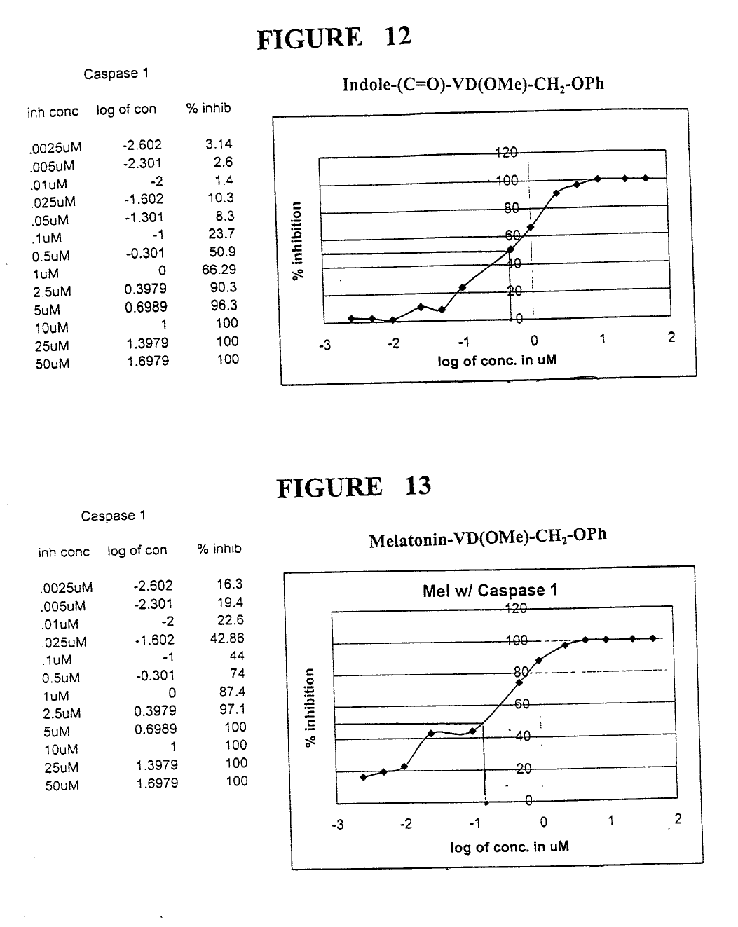

FIG. 12 is a graphic representation of indole-(2-carbonyl)-3-V-D(OMe)—CH₂—O—Ph with caspase 1 showing the log of the concentration in μM versus % inhibition. The antilog of +0.33 is 0.46. The IC₅₀ is approximately 0.46 μM.

FIG. 13 is a graphic representation of melatonin-V-D(OMe)—CH₂—O—Ph with caspase 1 showing the log of the concentration in μM versus % inhibition. The antilog of −0.857 is 0.139. The IC₅₀ is approximately 0.139 μM.

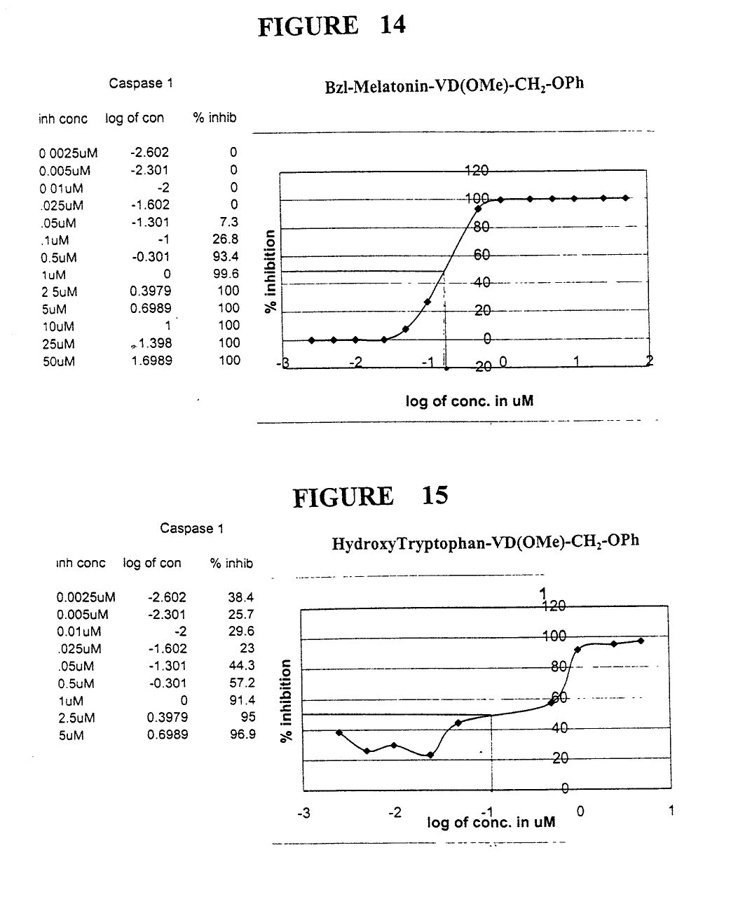

FIG. 14 is a graphic representation of Bzl-melatonin-V-D(OMe)—CH₂—O—Ph with caspase 1 showing the log of the concentration in μM versus % inhibition. The antilog of −0.7692 is 0.17. The IC₅₀ is approximately 0.17 μM.

FIG. 15 is a graphic representation of hydroxy Trp-TTP-V-D(OMe)—CH₂—O—Ph with caspase 1 showing the log of the concentration in μM versus % inhibition. The antilog of −0.939 is 0.115. The IC₅₀ is approximately 0.115 μM.

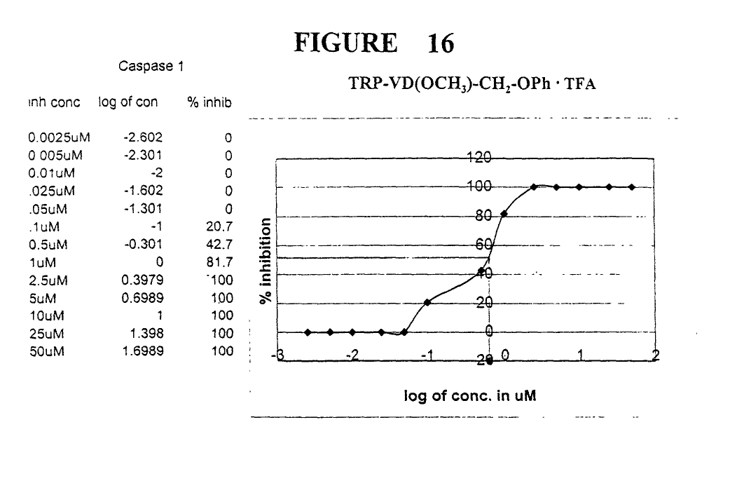

FIG. 16 is a graphic representation of TFA Trp-V-D(OMe)—CH₂—O—Ph TFA with caspase 1 showing the log of the concentration in μM versus % inhibition. The antilog of −0.205 is 0.624. The IC₅₀ is approximately 0.624 μM inhibits 50% of the caspase 1 activity.

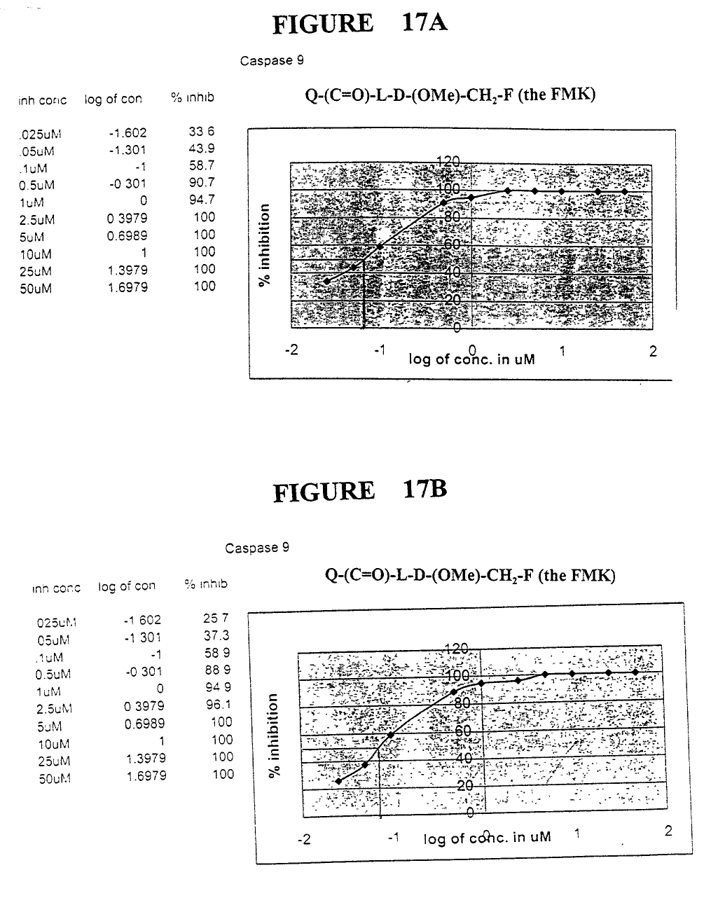

FIGS. 17A and 17B are graphic representations of non-esterase treated (17A) and esterase treated (17B) quinoline-(2-carbonyl)-L-D(OMe)—CH₂-FMK with caspase 9 showing the log of the concentration in μM versus % inhibition. The antilog of −1.146 is 0.065. The antilog of −1.146 is 0.07. The compound of FIG. 17A wherein the IC₅₀ is approximately at a concentration of 0.065 μM. The compound of FIG. 17B wherein the IC₅₀ is approximately 0.07 μM. Thus from the results, the esterase and non-esterase treated inhibitors have to have about the same inhibition properties.

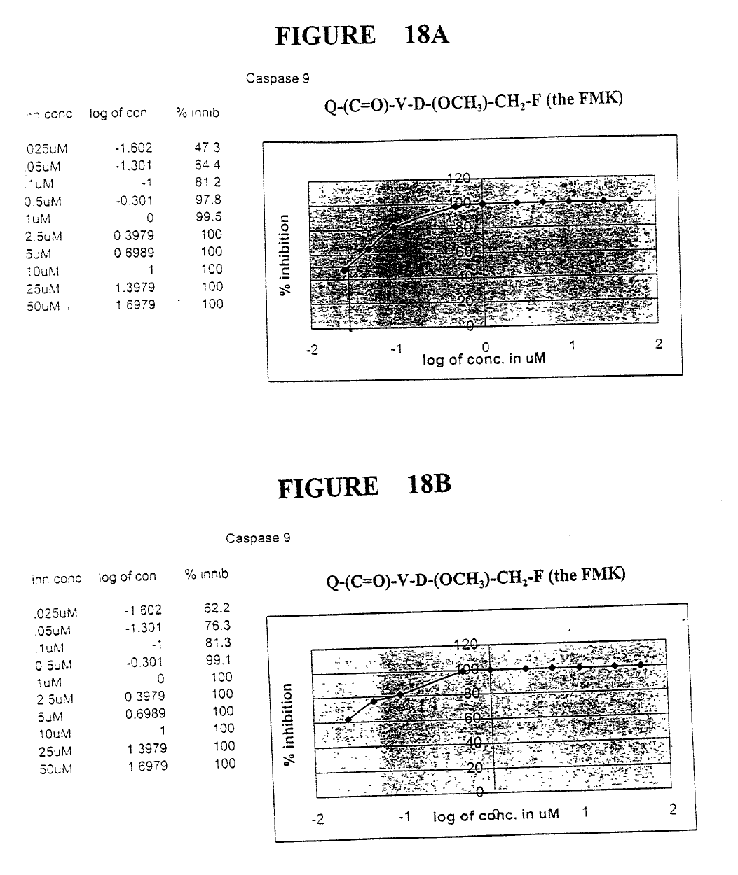

FIGS. 18A and 18B are graphic representations of non-esterase treated (18A) and esterase treated (18B) quinoline-(2-carbonyl)-V-D(OMe)—CH₂-FMK with caspase 9 showing the log of the concentration in μM versus % inhibition. For FIG. 18A, the antilog of −1.53 is 0.029. The compound of FIG. 18A wherein the IC₅₀ is approximately 0.029 μM inhibits 50% of the caspase 9 activity. The antilog −1.62 is 0.025. From FIG. 18B it appears that the esterase treated inhibitor Q-(C═O)-VD(OMe)-FMK is binding to the caspase 9 enzyme better than the non-treated inhibitor. This is the first observation of this result.

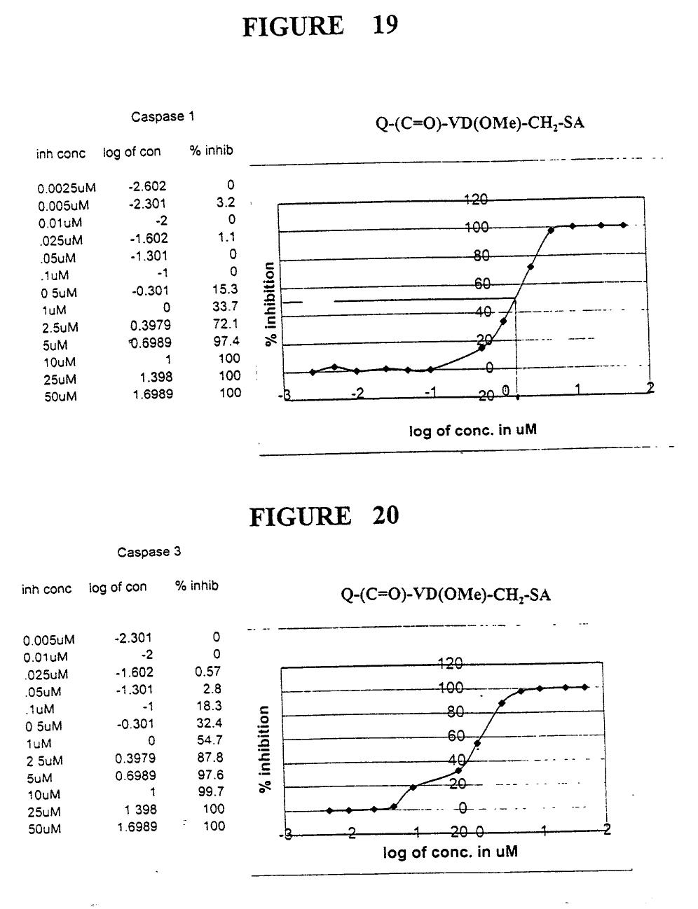

FIG. 19 is a graphic representation of quinoline-(2-carbonyl)-V-D(OMe)—CH₂-salicylic acid with caspase 1 showing the log of the concentration in μM versus % inhibition. The antilog of 0.1538 is −1.43. The IC₅₀ is approximately 1.43 μM.

FIG. 20 is a graphic representation of quinoline-(2-carbonyl)-V-D(OMe)—CH₂—(4-amino) salicylic acid with caspase 3 showing the log of the concentration in μM versus % inhibition. The antilog of −0.05 is 0.98. The IC₅₀ is approximately 0.98 μM.

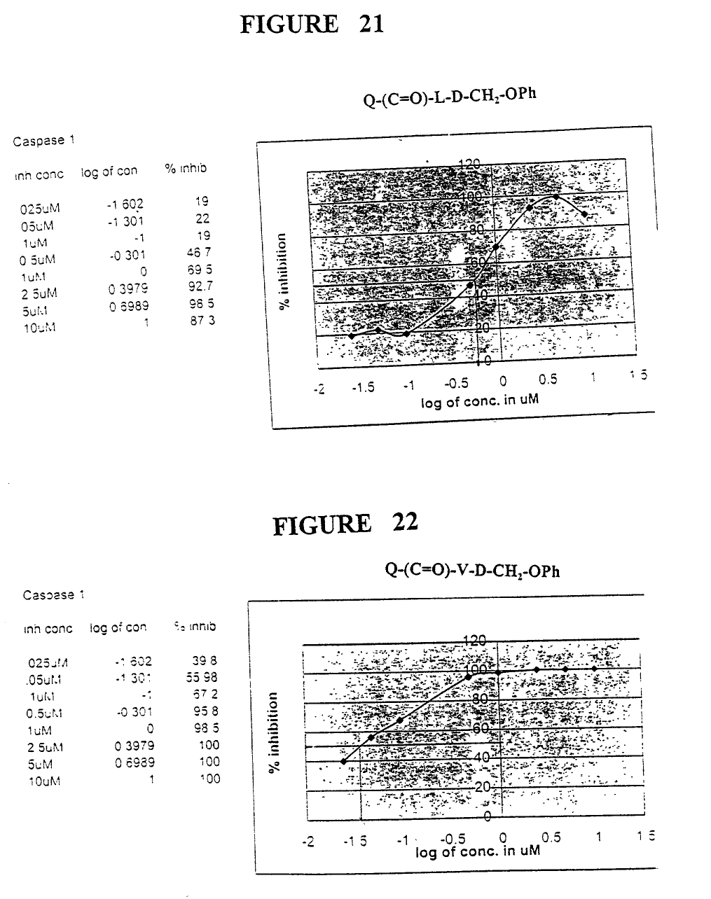

FIG. 21 is a graphic representation of quinoline-(2-carbonyl)-L-D(OCH₃)—CH₂—OPh with caspase 1 showing the log of the concentration in μM versus % inhibition. The antilog of −0.25 is 0.94. The IC₅₀ is approximately 0.94 μM.

FIG. 22 is a graphic representation of hydroxy quinoline-(2-carbonyl)-VD-OPh with caspase 1 showing the log of the concentration in μM versus % inhibition. The antilog of −1.423 is 0.038. The IC₅₀ is approximately 0.038 μM.

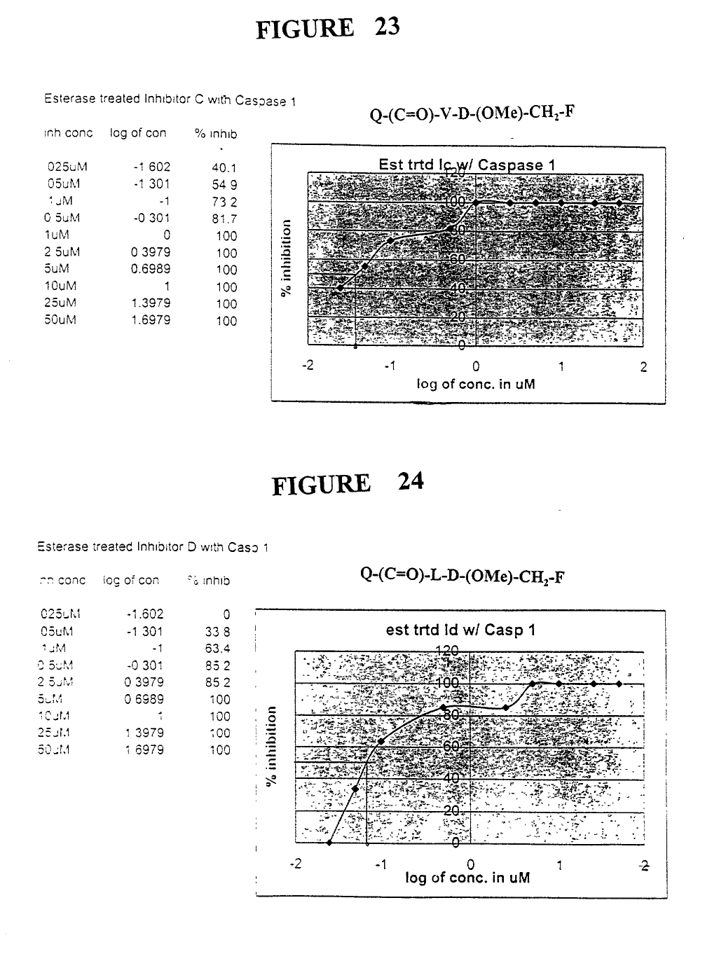

FIG. 23 is a graphic representation of esterase treated quinoline-(2-carbonyl)-L-D(OMe)-FMK with caspase 1 showing the log of the concentration in μM versus % inhibition. The antilog of −1.4 is 0.0398. The IC₅₀ is approximately 0.0398 μM.

FIG. 24 is a graphic of esterase treated quinoline-(2-carbonyl)-V-D(OMe)-FMK with caspase 1 showing the log of the concentration in μM versus % inhibited. The antilog of −1.168 is 0.068. The IC₅₀ is approximately of 0.068 μM.

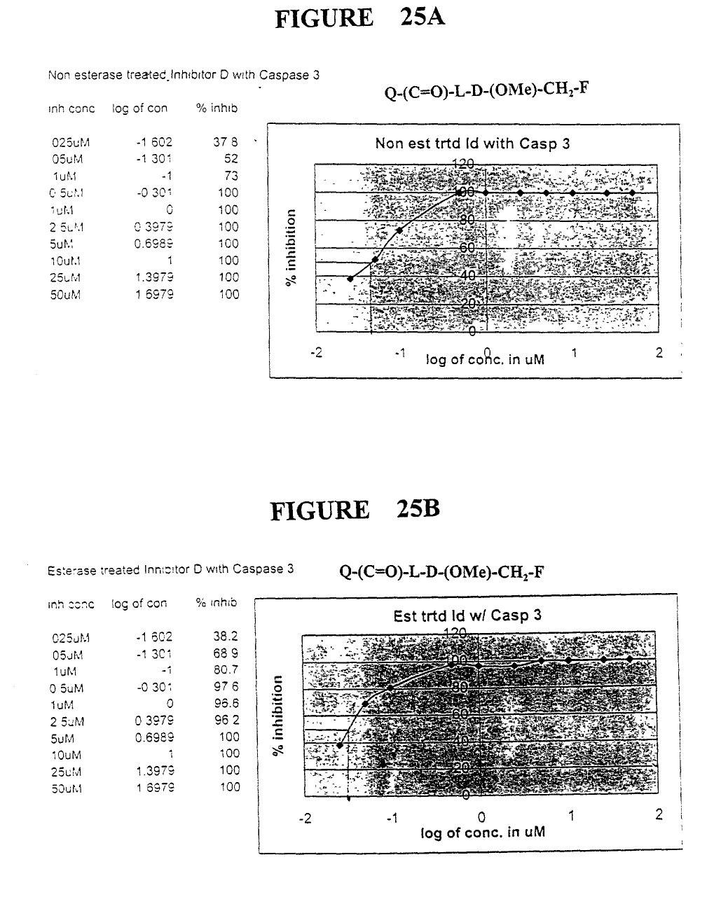

FIGS. 25A and 25B are graphic representations of non-esterase treated (25A) and esterase treated (25B) quinoline-(2-carbonyl)-L-D(OMe)-FMK with caspase 3 showing the log of the concentration in μM versus % inhibition. For FIG. 25A, the antilog of −1.346 is 0.045. This compound at a concentration of 0.045 μM inhibits 50% of the caspase 3 activity. In FIG. 25B, the antilog of −1.508 is 0.031. The IC₅₀ is approximately 0.031 μM inhibits. Thus the esterase treatment has a minor effect.

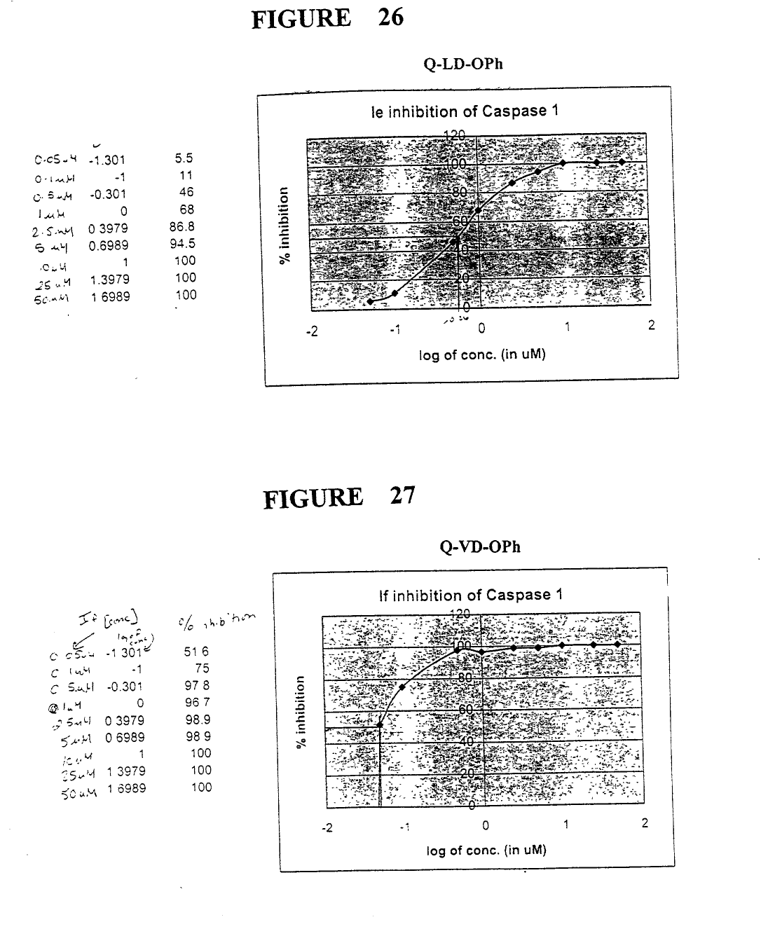

FIG. 26 is a graphic representation of quinoline-(2-carbonyl)-L-D-CH₂—OPh with caspase 1 showing the log of the concentration in μM versus % inhibition. The antilog is about 0.548. The IC₅₀ is approximately 0.548 μM.

FIG. 27 is a graphic representation of quinoline-(2-carbonyl)-V-D-CH₂—OPh with caspase 1 showing the log of the concentration in μM versus % inhibition. The antilog is about 0.05. The IC₅₀ is approximately 0.05 μM.

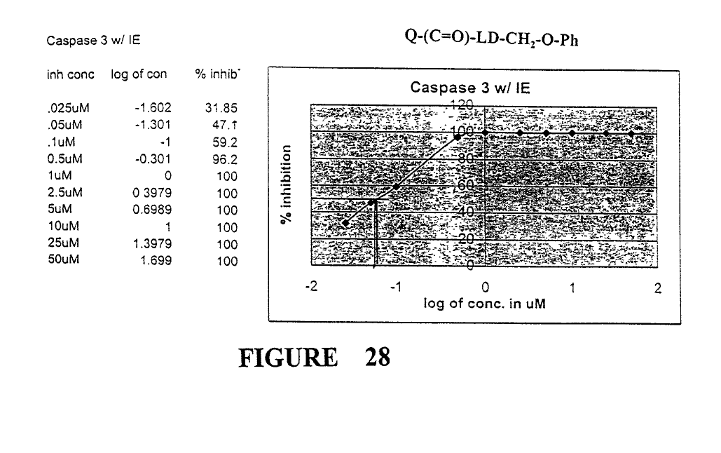

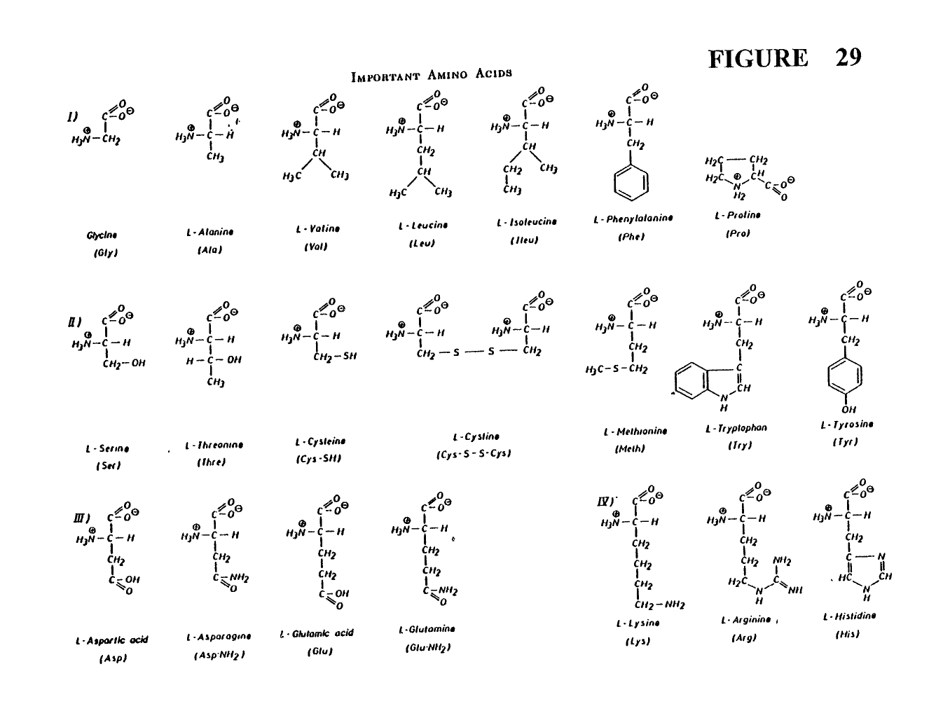

FIG. 28 is a graphic representation of quinoline-(2-carbonyl)-L-D-CH₂—OPh with caspase 3 showing the log of the concentration in μM versus % inhibition. The antilog of −1.255 is 0.056. The IC₅₀ is approximately 0.05 μM.

### Conclusions

Testing 10 different sequence in a blind test by TUNEL method provided the following conclusions:

a) The most efficacious sequences are: Q-AD(OMe)—CH₂-FMK, Q-VD(OMe)—CH₂—OPh, and Q-LD(OMe)—CH₂—OPh.

b) The order of efficiency (μm most to least) is:

Q-VD(OMe)—OPh. Q-LD(OMe)-Oph>Q-AD(OMe)-FMK.

ZVAD—FMK is effective at 20 μM.

Inhibitor (Structure) A¹ is effective at 40 μM.

Inhibitor B¹ is not effective.

Inhibitor C¹ is effective at 100 μM.

Inhibitor D¹ is effective at 20 μM.

Inhibitor E¹ is effective at 20 μM.

Inhibitor F¹ is not effective.

Inhibitor G¹ is effective at 100 μM.

Inhibitor H¹ is not effective.

Inhibitor I¹ is effective at 10 μM.

Inhibitor J¹ is effective at 10 μM.

### EXAMPLE 22

### Quinoline-(C═O)Val-Ala-Asp(OMe)-Fluoromethyl Ketone (Quinoline-VAD(OMe)-FMK

(a) To Z-VAD (OMe)-FMK (75 mg; 0.00016 mol) was added 30% HBr/AcOH. The reaction mixture was stirred for 30 min and evaporated to dryness to give the Hbr salt. To the solution of HBr salt in DMF (3 mL) was added 2-quinaldic acid (28 mg; 0.00016 mol), HOBT (0.00017 mol), HBTU (64 mg; 0.00017 mol) and DIEA (111 μl; 0.0006 mol). The reaction mixture was stirred for 3 hours and extracted with ethyl acetate. The ethyl acetate and aqueous fractions were separated. The EtOAc fraction was washed with 10% aqueous HCl, water, saturated aqueous NaHCO₃ solution, water, dried over anhydrous MgSO₄ and evaporated to give crude product. The product was purified by column chromatography over silica gel (230-400 mesh). Elution with EtOAc gave pure fractions which were mixed and evaporated to dryness to give product yield of 26 mg (33%). MS(EI):M⁺+489.2.

The concentration vs. >90% cell survival for QVAD(OMe)FMK is 100 μM.

(b) Similarly when Example 22 is repeated except that the Z-VAD(OMe) FMK is replace with a stoichiometrically equivalent amount of Z-VLD(OMe)FMK. A corresponding yield of the desired product is obtained.

(c) Similarly when Example 22(a) is repeated except that Z-VAD(OMe)FMK is replaced with Z-VAD(OMe)CH₂—2-(2-oxy-2,6-difluorophenyl) methyl ketone. A corresponding yield of the desired product is obtained.

### EXAMPLE 23

### Synthesis of 2-Quinoline-(C═O)-Val-Ala-Asp(OMe)-α-(2-Oxy-2,6-Difluorophenyl) Methyl Ketone

Boc-asp(OMe)-α-(2-oxy-2,6-difluorophenyl) methyl ketone (345 mg, 0.93 mmol) was dissolved in 95% TFA (4 ml), stirred for 1 hr, stripped under reduced pressure, chased with hexanes (3×5 ml) and pumped dry. To this solution in DMF (7 ml) were added 2-quinoline-(C═O)-val-ala-OH (320 mg, 0.93 mmol), HOBT (125 mg, 0.93 mmol), HBTU (351 mg, 0.93 mmol), followed by DIEA (0.48 ml, 2.8 mmol). The reaction mixture was stirred for 1 hr and EtOAc (50 ml) was added. The EtOAc and aqueous fractions were separated. The EtOAc fraction was washed with water, NaHCO₃ solution, NaCl solution, dried over MgSO₄ and the solvent removed. The residue was purified by column chromatograph on silica gel (eluting with 75% EtOAc in hexanes). Yield: 0.31 g (56%). TLC: R_{f} (100% ethyl acetate)=0.52. MS(EI): MH⁺=600.

### EXAMPLE 24

### Synthesis of 2-Quinoline-Asp(OMe)-Glu(OMe)-Val-OH

To a solution of H-glu(OMe)-val-O-t-Bu (525 mg, 1.66 mmol) in DMF (10 mol were added 2-quinoline-(C═O)-asp(OMe)—OH (500 mg, 1.66 mmol), HOBT (224 mg, 1.66 mmol), HBTU (630 mg, 1.66 mmol) followed by DIEA (0.86 ml, 4.98 mmol). The reaction mixture was stirred for 1 hr and EtOAc (100 ml) was added. The EtOAc and aqueous fractions were separated. The EtOAc fraction was washed with water, NaHCO₃ solution, NaCl solution, and dried over MgSO₄. The solvent was removed. The residue was purified by column chromatograph on silica gel (eluting with 100% EtOAc). Yield: 0.74 g (74%). The t-Butyl ester (700 mg, 1.17 mmol) was dissolved in 95% TFA (2 mol), stirred for 1 hr, stripped down, chased with hexanes (3×5 ml) and pumped dry. Yield: 0.6 g (94%). MS(EI): MH⁺=545.

### EXAMPLE 25

### Synthesis of 2-Quinoline-(C═O)-Asp(OMe)-Glu(OMe)-Val-Asp(OMe)-α-(2-oxy-2,6-Difluorophenyl) Methyl Ketone

Boc-asp(Ome)-α-(2-oxy-2,6-difluorophenyl) methyl ketone (345 mg, 0.93 mmol) was dissolved in 95% TFA (4 ml), stirred for 1 hr., stripped down, chased with hexanes (3×5 ml) and pumped dry. To this solution in DMF (7 ml) were added 2-quinoline-(C═O)-asp(OMe)-glu(OMe)-val-OH (505 mg, 0.93 mmol), HOBT (125 mg, 0.93 mmol), HBTU (351 mg, 0.93 mmol), followed by DIEA (0.48 ml, 2.8 mmol) and stirred for 1 hr. EtOAc (50 ml) was added. The EtOAc and aqueous fractions were separated. The EtOAc fraction was washed with water, NaHCO₃ solution, NaCl solution and dried over MgSO₄. The solvent was removed. The residue was purified by column chromatograph on silica gel (eluting with 100% EtOAc). Yield: 0.20 g (27%). TLC: R_{f}(100% ethyl acetate)=0.42. MS(EI): MH⁺=800.

### EXAMPLE 26

### Synthesis of 2-Quinoline-(C═O)-Asp(OMe)-Glu(OMe)-Val-Asp(OMe-Fluoromethyl Ketone

(a) Boc-asp(OMe)-FMK (245 mg, 0.93 mmol) was dissolved in 95% TFA (3 ml), stirred for 1 hr., stripped down under vacuum, chased with hexanes (3×5 ml) and pumped dry under vacuum. To this solution in DMF (7 ml) were added 2-quinoline-(C═O)-asp(OMe)-glu(OMe)-val-OH (505 mg, 0.93 mmol), followed by DIEA (0.48 ml, 2.8 mmol. The reaction mixture was stirred for 1 hr. EtOAc (50 ml) was added. The EtOAc and water layers were separated. The EtOAc fraction was washed with water, NaHCO₃ solution, NaCl solution, and dried over MgSO₄. The solvent was removed. The residue was purified by column chromatograph on silica gel (eluting with 100% EtOAc). Yield: 0.34 g (55%). MS(EI): MH⁺=690.

(b) Similarly Example 26(a) is repeated except that Boc-asp(OMe)-FMK is replaced with stoichiometrically equivalent amount Boc-asp(OMe)α-(2-oxy-2,6-difluorophenyl) methyl ketone. A corresponding yield of the desired compound is obtained.

(c) Similarly Example 26(a) is repeated except that the 2-quinoline (C═O)asp(OMe)-glu(OMe)val-OH is replaced by a stoichiometrically equivalent amount of 2-quinoline (C═O)-asp(OMe)-glu(OMe)-leu-OH. A corresponding yield of the desired compound is obtained.

While only a few embodiments of the invention have been shown and described herein, it will become apparent to those skilled in the art that various modifications and changes can be made in the quinoline-(2-carbonyl)-(multiple amino acid-amino acid-leaving group structures and quinoline-type structures) as pro-drugs and as protease inhibitors, their synthesis and their many pharmaceutical uses without departing from the spirit and scope of the present invention. All such modifications and changes coming within the scope of the appended claims are intended to be carried or thereby.

We claim:

1. A compound of the structure: 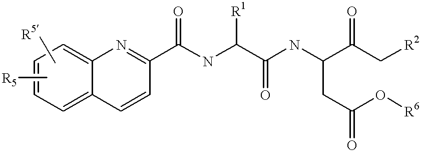
wherein in Structure I
R¹ is selected from the group consisting of alkyl, substituted alkyl, aryl, and substituted aryl which group —N—CH—(R¹)—(C═O)— will produce a natural amino acid structure or an unnatural amino acid structure, and;
the carbon adjacent to R¹ group is in the D or L configuration;
R² is selected from the group consisting of
—F;
and 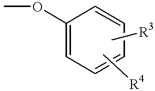
wherein R³ and R⁴ are each independently selected from the group consisting of hydrogen, alkyl, fluoro, chloro, carboxyl, alkoxy, alkyl carbonyl, aryl carbonyl, and amino;
and R⁵ and R⁵ are each independently selected from hydrogen, alkyl, alkoxy, fluoro, chloro, carboxy, alkyl carbonyl, aryl carbonyl, amino and together form a cyclic structure or a heterocyclic structure; and
R⁶ is selected from alkyl having 1 to 10 carbon atoms, aryl or substituted aryl; 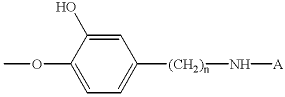
wherein A is a covalently bonded amine protecting group, and n is 1-4; 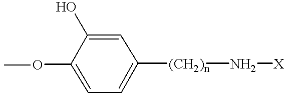
wherein X is a pharmaceutically acceptable salt, and n is 1-4; or 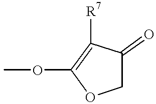
;
wherein R⁷ is selected from the group consisting of alkyl having 1 to 10 carbon atoms, aryl and alkylaryl.

2. A pharmaceutical composition for use as a protease inhibitor, which composition comprises
(a) a compound of the structure: 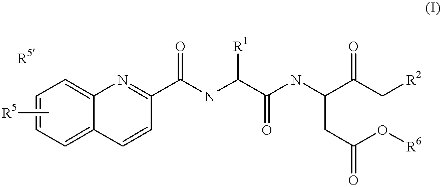
wherein in Structure I
R¹ is selected from the group consisting of alkyl, substituted alkyl, aryl, and substituted aryl which group —N—CH(R¹)—(C═O)— will produce a natural amino acid structure or an unnatural amino acid structure, and;
the carbon adjacent to R¹ group is in the D or L configuration;
R² is selected from the group consisting of
—F;
and 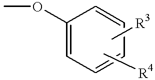
wherein R³ and R⁴ are each independently selected from the group consisting of hydrogen, alkyl, fluoro, chloro, carboxyl, alkoxy, alkyl carbonyl, aryl carbonyl, and amino;
and R⁵ and R⁵ are each independently selected from hydrogen, alkyl, alkoxy, fluoro, chloro, carboxy, alkyl carbonyl, aryl carbonyl, amino, and together can form a cyclic ring structure in a heterocyclic ring structure; and
R⁶ is selected from alkyl having 1 to 10 carbon atoms, aryl or substituted aryl; 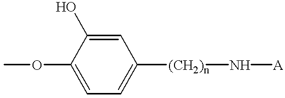
wherein A is a covalently bonded amine protecting group, and n is 1-4; 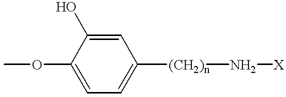
where X is the pharmaceutically accepted salt, and n is 1-4; 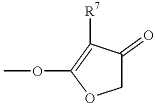
wherein R⁷ is selected from the group consisting of alkyl having 1 to 10 carbon atoms, aryl and alkylaryl or the pharmaceutically acceptable acid or base salts thereof, and
(b) a pharmaceutically acceptable excipient.

3. The composition of claim 2 wherein in the structure:
R¹ is selected from isopropyl or isobutyl;
R² is F ; and R⁵ is hydrogen.

4. The pharmaceutical composition of claim 2 wherein in the structure:
R¹ is selected from isopropyl or isobutyl;
R² is 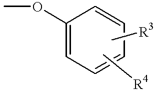
wherein R³ and R⁴ are each fluoro; and R⁵ is hydrogen.

5. The composition of claim 4 wherein in the structure, R³ and R⁴ in the 2 and 6 positions of the phenyl ring.

6. The composition of claim 5 wherein R² is

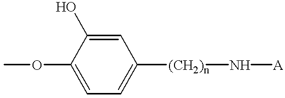

7. The composition of claim 5 wherein R² is

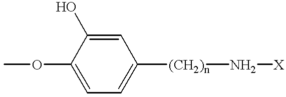

8. The composition of claim 5 wherein R² is

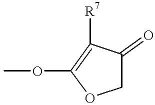

9. A pharmaceutical composition for use as a protease inhibitor, which composition comprises,
(a) a compound of the structure: 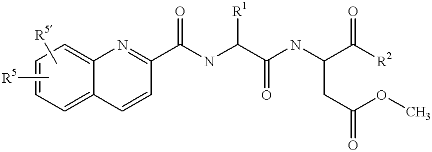
wherein
R¹ is selected from the group consisting of methyl, ethyl, isopropyl, and iso-butyl;
R² is selected from the group consisting of:
—F
or 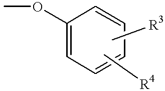
wherein R³ and R⁴ are each independently selected from the group consisting of hydrogen, alkyl having 1 to 10 carbon atoms, fluoro, chloro and amino;
and R⁵ and R⁵¹ are each selected from the group consisting of hydrogen having 1 to 10 carbon atoms, alkyl having 1 to 10 carbon atoms, alkoxyl having 1 to 10 carbon atoms, fluoro, and chloro; 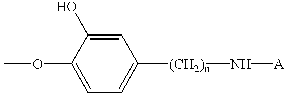
wherein A is a covalently bonded amine protecting group, and n is 1-4; 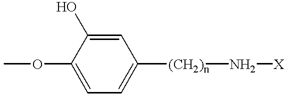
wherein X is a pharmaceutically acceptable salt and n is 1-4; 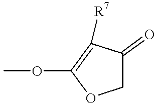
wherein R⁷ is selected from the group consisting of alkyl having 1 to 10 carbon atoms, aryl and alkylaryl.

10. The composition of claim 9 wherein R² is

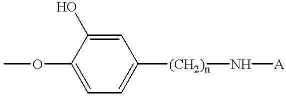

11. The composition of claim 9 wherein R² is

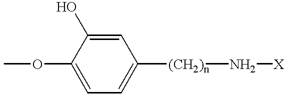

12. The composition of claim 9 wherein R² is

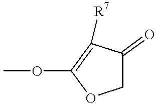

13. The pharmaceutical composition of claim 9, wherein in the structure:
R¹ is selected from isopropyl or iso-butyl;
R² is —F; and
R⁵ is hydrogen.

14. The pharmaceutical composition of claim 9 wherein, in the structure
R¹ is selected from isopropyl or isobutyl;
R² is 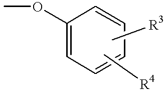
wherein R³ and R⁴ are each fluoro; and R⁵ is hydrogen.

15. The pharmaceutical composition of claim 9 wherein in the structure, groups R³ and R⁴ are in the 2 and 6 positions of the phenyl ring.

16. A pharmaceutical composition for use as an inhibitor to caspase or a caspase-like enzyme, which composition comprises
(a) a compound selected from the group consisting of: 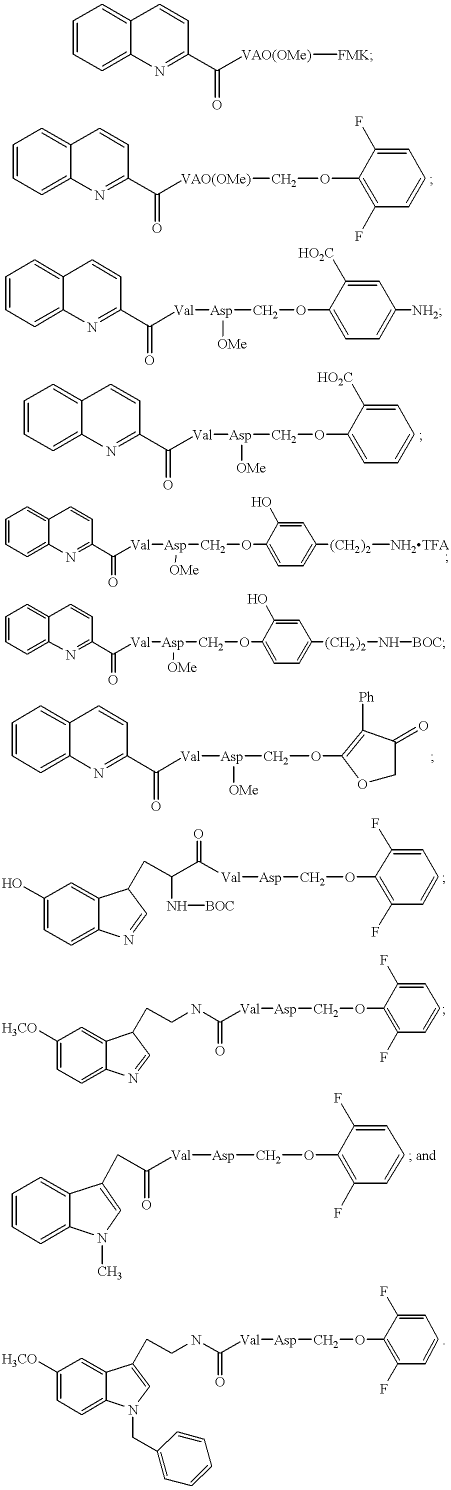
(b) a pharmaceutically acceptable excipient.

17. A compound of the structure: 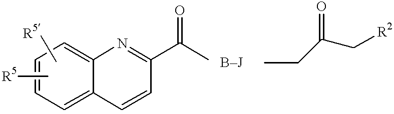
wherein
B and J are each selected from the group consisting of a natural amino acid structure or an unnatural amino acid structure, and;
the amino acid in the D or L configuration;
R² is selected from the group consisting of
—F
and 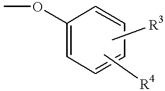
wherein R³ and R⁴ are each selected from the group consisting of hydrogen alkyl, fluoro, chloro, carboxyl, alkoxy, alkyl carbonyl, aryl carbonyl, and amino; and
R⁵ is selected from hydrogen, alkyl, alkoxy, fluoro, chloro, carboxy, alkoxy, alkyl carbonyl, aryl carbonyl, and amino.

18. The compound of claim 17 wherein groups B and J are both glycine and R² is fluoro and R⁵ is hydrogen.

19. A compound selected of the structure: 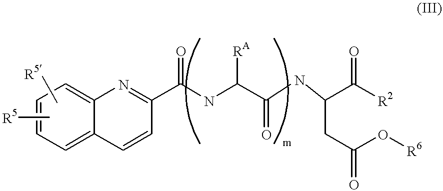
wherein in Structure III:
m is 1, 2 or 3, creating 1, 2 or 3 amino acid linkages, such that
when m=1, R^{A}=R¹,
when m=2, R^{A} is R¹ and R^{1B} in the separate amino acids and
when m=3, R^{A} is R¹, R^{1B} and R^{1C} wherein R¹, R^{1B} and R^{1C} in the separate amino acids which amino acids are the same or different amino acid when R¹, R^{1B} and R^{1C} are independently selected from the group consisting of alkyl, substituted alkyl, aryl, and substituted aryl which group —N—CH(R¹)—(C═O)—N—CH(R¹)—(C═O)—NH—CH(R^{1B})—(C═O); or NCH(R¹)(C═O)—NH—CH(R^{1B})(C═O)—NHCH(R^{1C})(C═O)— produces natural amino acid structures or an unnatural amino acid structures, and;
the carbon adjacent to R¹ group is in the D or L configuration;
R² is selected from the group consisting of:
—F;
and 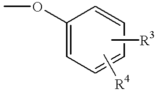
wherein R³ and R⁴ are each independently selected from the group consisting of hydrogen, alkyl, fluoro, chloro, carboxyl, alkoxy, alkyl carbonyl, aryl carbonyl, and amino;
and R⁵ and R^{5′} are each independently selected from hydrogen, alkyl, alkoxy, fluoro, chloro, carboxy, alkyl carbonyl, aryl carbonyl, amino and together form a cyclic ring structure or a heterocyclic ring structure; and
R⁶ is selected from alkyl having 1 to 10 carbon atoms, aryl or substituted aryl; 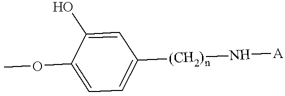
wherein A is a covalently bonded amine protecting group, and n is 1-4, preferably 2; 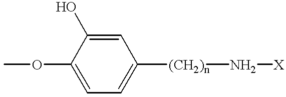
where X is the pharmaceutically accepted salt, and n is 1-4, preferably 2; and 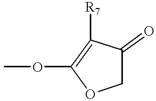
wherein R⁷ is selected from the group consisting of alkyl having 1 to 10 carbon atoms, aryl and alkylaryl or the pharmaceutically acceptable acid or base salts thereof.

20. The compound of claim 19 wherein m=2, R¹ and R^{1B} are each independently selected from methyl, ethyl, isopropyl and t-butyl.

21. The compound of claim 19 wherein m=3 , R¹, R^{1B} and R^{1C} are each independently selected from methyl, ethyl, isopropyl and t-butyl.

22. The compound of claim 20 wherein R² is F or 2,6-difluorophenoxy, R⁵ and R^{5′} are each hydrogen and R⁶ is methyl.

23. The compound of claim 21 wherein R² is F or 2,6-difluorophenoxy, R⁵ and R^{5′} are each hydrogen and R⁶ is methyl.

24. A pharmaceutical composition for use as a protease inhibitor having a compound selected from the structure: 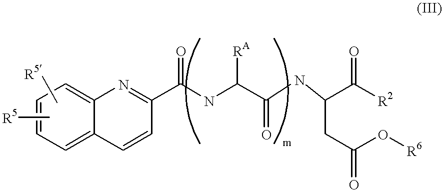
wherein in Structure III:
m is 1, 2 or 3, creating 1, 2 or 3 amino acid linkages, such that
when m=1, R^{A}=R¹,
when m=2, R^{A} is R¹ and R^{1B} in the separate amino acids and
when m=3, R^{A} is R¹, R^{1B} and R^{1C} wherein R¹, R^{1B} and R^{1C} in the separate amino acids which amino acids are the same or different amino acid when R¹, R^{1B} and R^{1C} are independently selected from the group consisting of alkyl, substituted alkyl, aryl, and substituted aryl which group —N—CH(R¹)—(C═O)—N—CH(R¹)—(C═O)—NH—CH(R^{1B})—(C═O); or NCH(R¹)(C═O)—NH—CH(R^{1B})(C═O)—NHCH(R^{1C})(C═O)— produces natural amino acid structures or an unnatural amino acid structures, and;
the carbon adjacent to R¹ group is in the D or L configuration;
R² is selected from the group consisting of:
—F;
and 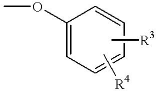
wherein R³ and R⁴ are each independently selected from the group consisting of hydrogen, alkyl, fluoro, chloro, carboxyl, alkoxy, alkyl carbonyl, aryl carbonyl, and amino;
and R⁵ and R^{5′} are each independently selected from hydrogen, alkyl, alkoxy, fluoro, chloro, carboxy, alkyl carbonyl, aryl carbonyl, amino and together form a cyclic ring structure or a heterocyclic ring structure; and
R⁶ is selected from alkyl having 1 to 10 carbon atoms, aryl or substituted aryl; 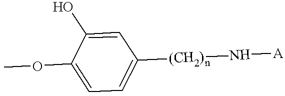
wherein A is a covalently bonded amine protecting group, and n is 1-4, preferably 2; 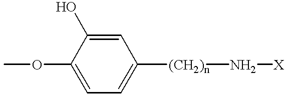
where X is the pharmaceutically accepted salt, and n is 1-4, preferably 2; and 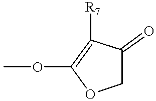
wherein R⁷ is selected from the group consisting of alkyl having 1 to 10 carbon atoms, aryl and alkylaryl or the pharmaceutically acceptable acid or base salts thereof; and a pharmaceutically acceptable excipient.

25. The pharmaceutical composition of claim 24 wherein m =2, R¹ and R^{1B} are each independently selected from methyl, ethyl, isopropyl and t-butyl.

26. The pharmaceutical composition of claim 24 wherein m=3 , R¹, R^{1B} and R^{1C} are each independently selected from methyl, ethyl, isopropyl and t-butyl.

27. The pharmaceutical composition of claim 25 wherein R² is F or 2,6-difluorophenoxy, R⁵ and R^{5′} are each hydrogen and R⁶ is methyl.

28. The pharmaceutical composition of claim 26 wherein R² is F or 2,6-difluorophenoxy, R⁵ and R^{5′} are each hydrogen and R⁶ is methyl.

29. A method of treatment of a human being diagnosed as having arthritis, metastases, infectious diseases, meningitis, salpingitis, septic shock, respiratory diseases, inflammatory condition, cholangitis, colitis, encephalitis, endocerolitis, hepatitis, pancreatitis, reperfusion injury, ischemic diseases, myocardial infarction, stroke, ischemic kidney disease immune-based diseases, hypersensitivity, auto-immune diseases, multiple sclerosis, bone diseases; and neurodegenerative diseases, Alzheimer's, Amyltrophic Lateral Sclerosis (ALS), Huntington's disease, Parkinson's disease, meningitis, spinal chord injuries and liver damage, traumatic brain injury, alopecia, AIDS and toxin induced liver disease, which method comprises:
A. Administering a therapeutically effective amount of the pharmaceutical composition of claim 2.

30. A method of treatment of a human being diagnosed as having arthritis, metastases, infectious diseases, meningitis, salpingitis, septic shock, respiratory diseases, inflammatory condition, cholangitis, colitis, encephalitis, endocerolitis, hepatitis, pancreatitis, reperfusion injury, ischemic diseases, myocardial infarction, stroke, ischemic kidney disease immune-based diseases, hypersensitivity, auto-immune diseases, multiple sclerosis, bone diseases; and neurodegenerative diseases, Alzheimer's, Amyltrophic Lateral Sclerosis (ALS), Huntington's disease, Parkinson's disease, meningitis, spinal chord injuries and liver damage, traumatic brain injury, alopecia, AIDS and toxin induced liver disease, which method comprises:
A. Administering a therapeutically effective amount of the pharmaceutical composition of claim 9.

31. A method of treatment of a human being diagnosed as having arthritis, metastases, infectious diseases, meningitis, salpingitis, septic shock, respiratory diseases, inflammatory condition, cholangitis, colitis, encephalitis, endocerolitis, hepatitis, pancreatitis, reperfusion injury, ischemic diseases, myocardial infarction, stroke, ischemic kidney disease immune-based diseases, hypersensitivity, auto-immune diseases, multiple sclerosis, bone diseases; and neurodegenerative diseases, Alzheimer's, Amyltrophic Lateral Sclerosis (ALS), Huntington's disease, Parkinson's disease, meningitis, spinal chord injuries and liver damage, traumatic brain injury, alopecia, AIDS and toxin induced liver disease, which method comprises:
A. Administering a therapeutically effective amount of the pharmaceutical
A. Administering a therapeutically effective amount of the pharmaceutical composition of claim 24.

35. A method of treatment of a human being diagnosed as having arthritis, metastases, infectious diseases, meningitis, salpingitis, septic shock, respiratory diseases, inflammatory condition, cholangitis, colitis, encephalitis, endocerolitis, hepatitis, pancreatitis, reperfusion injury, ischemic diseases, myocardial infarction, stroke, ischemic kidney disease immune-based diseases, hypersensitivity, auto-immune diseases, multiple sclerosis, bone diseases; and neurodegenerative diseases, Alzheimer's, Amyltrophic Lateral Sclerosis (ALS), Huntington's disease, Parkinson's disease, meningitis, spinal chord injuries and liver damage, traumatic brain injury, alopecia, AIDS and toxin induced liver disease, which method comprises:
A. Administering a therapeutically effective amount of the pharmaceutical composition of claim 25.

36. A method of treatment of a human being diagnosed as having arthritis, metastases, infectious diseases, meningitis, salpingitis, septic shock, respiratory diseases, inflammatory condition, cholangitis, colitis, encephalitis, endocerolitis, hepatitis, pancreatitis, reperfusion injury, ischemic diseases, myocardial infarction, stroke, ischemic kidney disease immune-based diseases, hypersensitivity, auto-immune diseases, multiple sclerosis, bone diseases; and neurodegenerative diseases, Alzheimer's, Amyltrophic Lateral Sclerosis (ALS), Huntington's disease, Parkinson's disease, meningitis, spinal chord injuries and liver damage, traumatic brain injury, alopecia, AIDS and toxin induced liver disease, which method comprises:
A. Administering a therapeutically effective amount of the pharmaceutical composition of claim 26.
